(19) United States
(12) Patent Application Publication
(10) Pub. No.: US 2002/0188016 A9
(43) Pub. Date: Dec. 12, 2002

(54) Heterocycle derivatives and methods of use

(76) Inventors: Johnny W. Peterson, Dickinson, TX (US); Deborah L. Gessell-Lee, Galveston, TX (US); Shamsher S. Saini, Dickinson, TX (US)

Correspondence Address: MUETING, RAASCH & GEBHARDT, P.A., P.O. BOX 581415, MINNEAPOLIS, MN, 55458, US

(21) Appl. No.: 09/860,652

(22) Filed: May 19, 2001

Related U.S. Application Data
(60) Provisional application No. 60/210,412, filed on Jun. 8, 2000.

(51) Int. Cl. A61K 31/5575; A61K 31/4164

(52) U.S. Cl. 514/396; 514/559

(57) ABSTRACT

The present invention provides methods for treating intestinal fluid loss, whooping cough, anthrax, and conditions associated with smooth muscle contraction. The present invention also provides methods for inhibiting adenylate cyclase in vivo and in vitro.

### CONTINUING APPLICATION DATA

This application claims the benefit of U.S. Provisional Application Serial No. 60/210,412, filed Jun. 8, 2000, which is incorporated by reference herein.

### GOVERNMENT FUNDING

The present invention was made with government support under Grant No. 2 R01 AI 21463, awarded by the National Institutes of Allergy and Infectious Diseases (NIAID); Grant No. 2 R01 AI 18401, awarded by the NIAID; Grant No. ES06676, awarded by the National Institute Environmental Health Sciences (NIEHS); and Grant No. R01 ES06839, awarded by the NIEHS. The Government may have certain rights in this invention.

### BACKGROUND

Diarrheal diseases in humans and non-human animals can be caused by several types of pathogens, including viruses, bacteria, parasites, and rotaviruses. The most prevalent are the bacteria Escherichia coli and Vibrio cholerea. Diarrheal diseases are a prevalent cause of morbidity and mortality in less developed countries. These diseases also afflict populations in developed countries. For example, each year in the US over 200,000 children 5 years and younger are hospitalized with acute diarrheal diseases. The infectious diarrheas are the leading cause of morbidity and mortality worldwide a common class of illness in the United States.

Due to its many causes, acute infectious diarrhea can occur more than once in the same person, and, therefore, it is unlike most chronic conditions which typically occur once. Unlike other digestive diseases, infectious diarrheas are communicable via person-to-person contact or through contaminated food or water and can spread endemically or in epidemics through households, schools, day-care centers, nursing homes, and communities. Diarrheal diseases also pose a serious challenge in the raising of non-human animals in the farming industry, particularly with young calves and pigs.

### SUMMARY OF THE INVENTION

The present invention represents an advance in the art of treating intestinal fluid loss in a subject. The invention provides methods for treating intestinal fluid loss in a subject. The method includes administering to a subject who has or is at risk of developing intestinal fluid loss a composition that includes an effective amount of heterocycle-containing compounds such as a heterocycle derivative, for instance a diphenyl heterocycle derivative, a prostaglandin analog, or a combination thereof. In some embodiments of this aspect of the invention the fluid loss is not associated with a pathogen polypeptide having ADP-ribosylation activity, and in other aspects the intestinal fluid loss is associated with a pathogen polypeptide having ADP-ribosylation activity.

The present invention represents an advance in the art of inhibiting adenylate cyclase. The ability of the compounds to inhibit adenylate cyclase was surprising and unexpected since some of the compounds were designed to specifically react with the active site of either cyclooxygenase 1 or cyclooxygenase 2. The present invention provides a method for inhibiting adenylate cyclase in vitro. The method includes contacting an adenylate cyclase with a composition containing an amount of a heterocycle-containing compound effective to inhibit the generation of adenosine 3′,5′-monophosphate (cAMP) from adenosine triphosphate (ATP). The adenylate cyclase may be in vivo, in which case the method includes contacting a cell that includes an adenylate cyclase with the composition. In some embodiments, the cell does not comprise a pathogen polypeptide having ADP-ribosylation activity. In these embodiments, the heterocycle-containing compound is preferably a diphenyl heterocycle derivative, a prostaglandin analog, or a combination thereof. In other embodiments, the cell includes a pathogen polypeptide having ADP-ribosylation activity.

Also provided by the invention is a method for inhibiting smooth muscle contraction in a subject. The method includes administering to a subject who has or is at risk of developing a condition associated with smooth muscle contraction a composition including an effective amount of a heterocycle derivative, for instance a diphenyl heterocycle derivative, a prostaglandin analog, or a combination thereof.

The present invention further provides a method for treating whooping cough in a subject, including administering to a subject who has or is at risk of developing whooping cough a composition that includes an effective amount of a heterocycle-containing compound.

The present invention also provides a method for treating anthrax in a subject, including administering to a subject who has or is at risk of developing anthrax a composition that includes an effective amount of a heterocycle-containing compound.

Unless otherwise specified, “a,” “an,” “the,” and “at least one” are used interchangeably and mean one or more than one.

### BRIEF DESCRIPTION OF THE FIGURES

FIG. 1. Inhibitory effect of histidine on fluid accumulation in mouse intestinal loops challenged with cholera toxin (1 μg) compared to control mice. The vertical bars indicate one standard error above and below the arithmetic means. The asterisk indicates a significant difference (P<0.05) as determined by Dunnett's Multiple Group Comparison test. The number of mice per group is indicated above each bar. CT control, mice receiving only cholera toxin (CT); CT+L-his(0.93 mg), mice receiving CT and 0.93 mg of L-histidine; CT+L-his(0.3.7 mg), mice receiving CT and 3.7 mg of L-histidine; and CT+L-his(14.8 mg), mice receiving CT and 14.8 mg of L-histidine.

FIG. 2. Normalized values of I_{SC} in Ussing chambers. Control, the tissue was bathed on both sides by NaCl solution; PGE₂, 1 μM PGE₂ was added to the basolateral solution, which stimulated Na⁺ transport and increased the steady-state short circuit current (I_{SC}) by 14% (the maximum PGE₂-induced change in I_{SC} was 18±3%, p<0.01); PGE₂+L-histidine, a 1 μM PGE₂+10 mM L-histidine solution was incubated at 37° C. for 30 minutes and then added to the basolateral side (the I_{SC} decreased to 30±9% of control); Difference, difference in I_{SC} between PGE₂ and PGE₂+L-histidine, which was 78±21% (p<0.025); I_{SC} (μA/cm²), short circuit current (microamperes per square centimeter).

FIG. 3. Panel A. C-18 reverse-phase separation of PGE₂ and adducts of PGE₂. Panel B. C-18 reverse-phase chromatography of [³H]-PGE₂ and imidazole. Imi, imidazole; PGE₂-IMI and PGE₂-Imi, PGE₂-imidazole.

FIG. 4. Inhibition of CT-induced cAMP formation with purified PGE₂-imidazole covalent adduct. The vertical bars represent standard error of the mean of triplicate samples from a typical experiment assayed in duplicate with a cAMP ELISA. The asterisk indicates statistical significance by Dunnett's Multiple Group Comparison test (P<0.05). cAMP (pmoles), picomoles cyclic AMP; CT, cholera toxin; PGE₂-imi, PGE₂-imidazole.

FIG. 5. Reduction of CT-induced fluid accumulation in murine intestinal loops by PGE₂-imidazole adduct. PGE₂-imidazole adduct was instilled into ligated intestinal loops at the time of challenge with CT (1 μg/loop). The amount of purified PGE₂-imidazole injected into each loop is indicated on the abscissa. Panel A—The mice were necropsied after a standard 6 hour incubation period, and fluid accumulation was measured. The vertical bars indicate one standard error above and below the arithmetic means derived from 5-8 mice per group. The asterisk indicates a significant difference (P<0.05) as determined by Dunnett's Multiple Group Comparison test. Panel B—Cyclic AMP levels in the intestinal fluids and PBS lavages of negative loops from the mice in Panel A were assayed by a cAMP ELISA. The vertical bars indicate one standard error above and below the arithmetic means derived from 5-8 mice per group.

FIG. 6. Formation of PGE₂-histidine covalent adducts when PGE₂ (4.7 mM) was mixed with 181 mM histidine. After incubation at 37° C. (pH 7.0) under N₂ for periods up to 24 hour, the reaction mixtures were separated by chromatography on a C-18 reverse-phase column eluted with 26% acetonitrile and 0.1% TFA. The area of the PGE₂-histidine peak (190 mm) migrating at 12.5 min was determined for each time period.

FIG. 7. Stability of the PGE₂-imidazole adduct. Minor peak I, peak I from FIG. 3A.

FIG. 8. Panel A. Electrospray-MS/MS daughter ion spectrum obtained from the pseudo-molecular ion at m/z 403 for the PGE₂-imidazole adduct. Panel B. Electrospray-MS/MS daughter ion spectrum obtained from the pseudo-molecular ion at m/z 419 for the methyl esterified PGE₂-imidazole (¹⁵N) adduct.

FIG. 9. (A) One-dimensional proton nuclear magnetic resonance (¹H NMR) spectrum, (B) 2 dimensional totally correlated spectroscopy (2D TOCSY) spectrum, and (C) 2D ¹⁵N-labeled proton hetereonuclear multiple bond coherence spectroscopy (¹⁵N/¹H HMBC spectrum) of PGE₂-imidazole adduct in D₂O at 600 MHz. In (C) F1 is the ¹⁵N dimension and F2 is the ¹H dimension.

FIG. 10. (A) Proposed mechanism for formation of PGE₂-imidazole adduct, (B) structures of PGB₂ and PGB₂-imidazole adduct.

FIG. 11. Celecoxib reduced CT-induced fluid accumulation in murine intestinal loops. CT, cholera toxin; CT+celecoxib in loop, mice challenged with cholera toxin and two 80 microgram (mg) doses of celecoxib (one injected into the intestinal lumen at the time of challenge with CT, the second injected intraperitoneally two hours later); CT+celecoxib IP only, mice challenged with cholera toxin and two 80 microgram (μg) doses of celecoxib (one injected intraperitoneally at the time of challenge with CT, the second injected intraperitoneally two hours later). The vertical bars indicate one standard error above or below the mean. The asterisks indicate a significant difference from the positive control group as determined by the Tukey test (P<0.05).

FIG. 12. Effect of imidazole (2.7 mmoles), PGE₂-Histidine adduct (52 μmoles) and celecoxib (0.52 mmoles) on the enzyme Adenylate Cyclase (4.6 nmoles). Blank has no enzyme and inhibitors, while Enzyme (E) has only enzyme and no inhibitors. Enzyme containing specific inhibitors are represented as E+imidazole, E+PGE₂-Histidine and E+celecoxib. Significant difference from the control value (E) is indicated by *P≦0.05 and *P≦0.001 as determined by Student's t-test.

FIG. 13. Fluid accumulation in Cholera toxin challenged murine intestinal ligated loops treated with the COX-1 inhibitor SC-560. n, number of animals; CT 1 μg/loop, 1 microgram of cholera toxin added to each loop; CT+9 nM SC-560, 1 microgram of cholera toxin and 9 nanomolar SC-560 added to each loop The asterisks indicate a significant difference from the positive control (CT) as determined by the Tukey test.

FIG. 14. IC₅₀ of PGE₂-histidine adduct for adenylate cyclase.

FIG. 15. IC₅₀ of celecoxib for adenylate cyclase.

FIG. 16. IC₅₀ of imidazole adduct for adenylate cyclase.

### DETAILED DESCRIPTION OF PREFERRED EMBODIMENTS OF THE INVENTION

The present invention provides methods that involve the use of compositions including a heterocycle-containing compound, particularly a heterocycle derivative. As used herein, a “heterocycle-containing” compound includes unsubstituted heterocyclic compounds (preferably, imidazole, pyrazole, thiophene, and furan), and more preferably, imidazole, as well as derivatives thereof. As used herein, a “heterocycle-containing compound” is a compound that includes a heterocyclic structure where 5 atoms make up the closed ring, and at least one of the 5 members of the ring is a heteroatom. The heteroatom is preferably nitrogen, oxygen, or sulfur. Preferably, the heterocycle-containing compound is a “heterocycle derivative” that includes a 5-membered core heterocyclic ring with at least one ring substituent. Examples of heterocycle-containing compounds that form the core structure of heterocycle derivatives include imidazole, pyrazole, thiophene, and furan.

Preferably, the heterocycle derivatives are substituted with at least one nonfused ring structure, preferably, a nonfused 5- or 6-membered ring, which can optionally be further substituted. This ring structure may or may not be bonded to a heteroatom in the core heterocyclic ring. The core heterocyclic ring can optionally be substituted with nonring substituents. Examples of such substituents include halogen atoms (preferably, Br), (C1-C4)alkyl groups (preferably, CH₃), perfluorinated (C1-C4)alkyl groups (preferably, CF₃), carbonyl groups, N₂O, (C1-C4)alkoxy groups (preferably, OCH₃), hydroxy substituted (C1-C4)alkyl groups (preferably, CH₂CH₂OH), carboxylic acid substituted (C1-C4)alkyl groups (preferably, CH₂COOH), and CH₂CH(NH₂)COOH.

If a substituted 5- or 6-membered ring is present in the heterocycle derivatives, it is substituted with halogen atoms (preferably, F or Cl), (C1-C4)alkoxy (preferably, —OCH₃), (C1-C4)alkyl groups (preferably, CH₃), a saturated or unsaturated (C1-C10)alkyl group, optionally substituted with hydroxyls, carbonyls, and/or carboxylic acids, or the following:

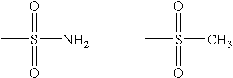

Preferred ring structures that are bonded to the core heterocyclic ring are as follows:

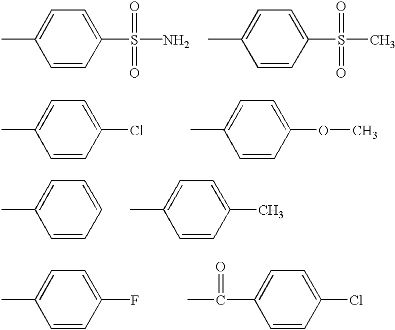

For certain preferred methods of the present invention, the ring structure is a prostaglandin. Such heterocycle derivatives are referred to herein as “prostaglandin analogs.” For certain other preferred methods of the present invention, the ring structure is a substituted or unsubstituted phenyl ring. For particularly preferred methods, the heterocycle derivative has two phenyl rings, which can be substituted or unsubstituted. Such heterocycle derivatives are referred to herein as “diphenyl heterocycle” derivatives. Preferably, both of the substituted or unsubsitituted phenyl rings are nonused rings. As such, in some aspects of the present invention, a diphenyl heterocycle does not include indomethacin, which has the following structure:

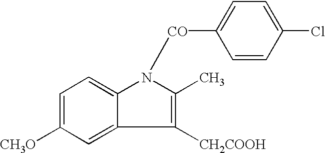

Preferred examples of diphenyl heterocycle derivatives include the following:

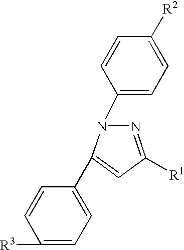

where R¹ is a perfluorinated (C1-C4)alkyl group (preferably, CF₃) or H; R² and R³ are each independently a halogen atom (preferably, F or Cl), (C1-C4)alkoxy (preferably, —OCH₃), (C1-C4)alkyl groups (preferably, CH₃), H, or

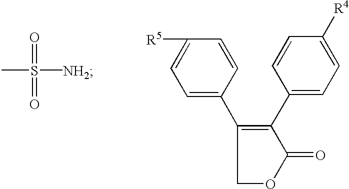

where R⁴ and R⁵ are each independently H, or a

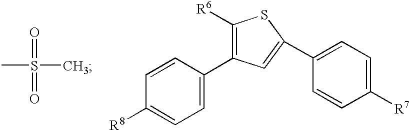

where R⁶ is a halogen atom (preferably, Br) or H; and where R⁷ and R⁸ are each independently a halogen atom (preferably, F), H, or

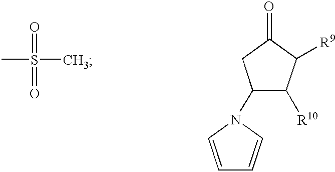

where R⁹ and R¹⁰ are each independently a saturated or unsaturated (C1-C10)alkyl group, optionally substituted with hydroxyls, carbonyls, and/or carboxylic acids. Preferably R⁹ is as follows:

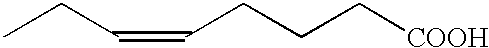

and R¹⁰ is as follows:

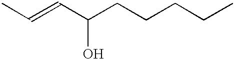

More preferred examples of diphenyl heterocycles include:

rofecoxib (available under the trade designation VIOXX, from Merck & Co., Whitehouse Station, N.Y.), which has the following structure:

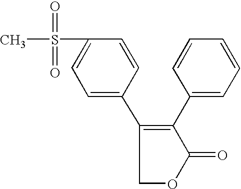

celecoxib (available under the trade designation CELEBREX, from Searle and Co., Skokie, Ill.), which has the following structure:

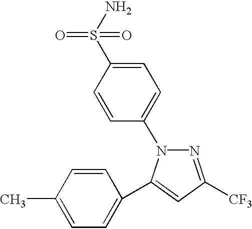

a compound available under the trade designation SC-560 from Cayman Chemical Co., Ann Arbor, Mich., which has the following structure:

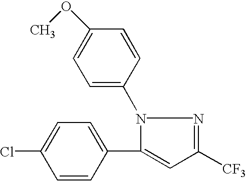

and a compound available under the trade designation DuP-697 from Cayman Chemical Co., which has the following structure:

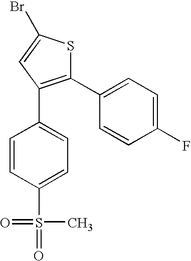

As used herein, the term “prostaglandin analog” refers to a type of heterocycle derivative that has, in addition to the core 5-membered heterocyclic ring, a prostaglandin. As used herein, a “prostaglandin” is a 20-carbon fatty acid, typically derived from arachidonic acid. Preferably, the prostaglandin is PGE₂, which has the following structure:

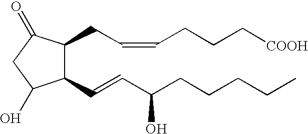

When the prostaglandin is PGE₂, preferably the heterocycle is covalently attached to the C11 of the prostaglandin. Preferred examples of prostaglandin analogs include prostaglandin E2-imidazole (PGE₂-imidazole) adduct, which has the structure:

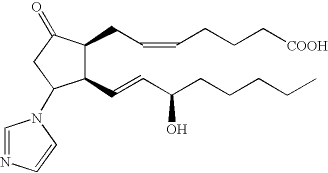

and prostaglandin E2-histidine (PGE₂-histidine) adduct, which has the structure:

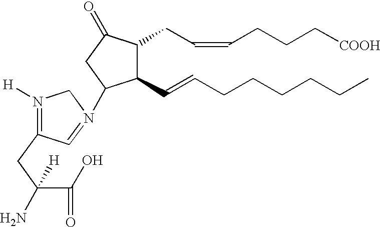

The prostaglandin analogs of the present invention can be produced by incubating a prostaglandin in the presence of the heterocycle that is to be covalently attached to the prostaglandin. Preferably, the prostaglandin used is PGE₂, PGA₂, or PGB₂. Prostaglandins can be obtained from Sigma Chemical Co., St. Louis, Mo. The conditions of incubation preferably include a temperature of from about 25° C. to about 40° C., more preferably about 37° C. The pH of the mixture is preferably greater than about pH 6.5, more preferably about pH 7.4. Optionally, the mixture may contain a buffer to maintain the desired pH. The incubation is preferably allowed to proceed for about 1 hour to about 24 hours, more preferably about 24 hours. Due to the tendency of prostaglandins to oxidize in the presence of oxygen, the reaction between a prostaglandin and a heterocycle is preferably conducted in the presence of an inert gas, such as nitrogen. Preferably, when the heterocycle to be added to a prostaglandin is histidine, L-histidine is used. The structure of the prostaglandin analog can be determined using methods known to the art including, for instance, mass spectrometry and nuclear magnetic resonance (NMR).

The compositions used in the methods of the present invention may further include a pharmaceutically acceptable carrier. Typically, the composition includes a pharmaceutically acceptable carrier when the composition is used as described below in “Methods of Use.” The compositions of the present invention may be formulated in pharmaceutical preparations in a variety of forms adapted to the chosen route of administration. Formulations include those suitable for oral, rectal, vaginal, intraintestinal, intramuscular, intraperitoneal, intranasal, intravenous, cervical or uterine implant, transmucosal, transdermal administration, or combinations thereof. Daily dosages of the compounds described herein are typically from about 1 mg/kg up to about 10 mg/kg.

The formulations may be conveniently presented in unit dosage form and may be prepared by methods well known in the art of pharmacy. All methods of preparing a pharmaceutical composition include the step of bringing the active compound (e.g., a heterocycle derivative) into association with a carrier that constitutes one or more accessory ingredients. In general, the formulations are prepared by uniformly and intimately bringing the active compound into association with a liquid carrier, a finely divided solid carrier, or both, and then, if necessary, shaping the product into the desired formulations.

Typically, the compositions of the invention will be administered from about 1 to about 5 times per day. The amount of active ingredient that may be combined with the carrier materials to produce a single dosage form will vary depending upon the subject treated and the particular mode of administration. A typical preparation will contain from about 5% to about 95% active compound (w/w). Preferably, such preparations contain from about 20% to about 80% active compound. The amount of heterocycle-containing compound in such therapeutically useful compositions is such that the dosage level will be effective to prevent or suppress the condition the subject has or is at risk for.

Formulations suitable for parenteral administration conveniently comprise a sterile aqueous preparation of the composition, or dispersions of sterile powders that include the composition, which are preferably isotonic with the blood of the recipient. Isotonic agents that can be included in the liquid preparation include sugars, buffers, and sodium chloride. Solutions of the composition can be prepared in water, and optionally mixed with a nontoxic surfactant. Dispersions of the composition can be prepared in water, ethanol, a polyol (such as glycerol, propylene glycol, liquid polyethylene glycols, and the like), vegetable oils, glycerol esters, and mixtures thereof. The ultimate dosage form is sterile, fluid and stable under the conditions of manufacture and storage. The necessary fluidity can be achieved, for example, by using liposomes, by employing the appropriate particle size in the case of dispersions, or by using surfactants. Sterilization of a liquid preparation can be achieved by any convenient method that preserves the bioactivity of the composition, preferably by filter sterilization. Preferred methods for preparing powders include vacuum drying and freeze drying of the sterile injectable solutions. Subsequent microbial contamination can be prevented using various antimicrobial agents, for example, antibacterial, antiviral and antifungal agents including parabens, chlorobutanol, phenol, sorbic acid, thimerosal, and the like. Absorption of the composition by the animal over a prolonged period can be achieved by including agents for delaying, for example, aluminum monostearate and gelatin.

Formulations of the present invention suitable for oral administration may be presented as discrete units such as tablets, troches, capsules, lozenges, wafers, or cachets, each containing a predetermined amount of the active compound as a powder or granules, as liposomes containing the heterocycle-containing compound, or as a solution or suspension in an aqueous liquor or non-aqueous liquid such as a syrup, an elixir, an emulsion or a draught.

The tablets, troches, pills, capsules, and the like may also contain one or more of the following: a binder such as gum tragacanth, acacia, corn starch or gelatin; an excipient such as dicalcium phosphate; a disintegrating agent such as corn starch, potato starch, alginic acid and the like; a lubricant such as magnesium stearate; a sweetening agent such as sucrose, fructose, lactose or aspartame; and a natural or artificial flavoring agent. When the unit dosage form is a capsule, it may further contain a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, or sugar and the like. A syrup or elixir may contain one or more of a sweetening agent, a preservative such as methyl- or propylparaben, an agent to retard crystallization of the sugar, an agent to increase the solubility of any other ingredient, such as a polyhydric alcohol, for example glycerol or sorbitol, a dye, and flavoring agent. The material used in preparing any unit dosage form is substantially nontoxic in the amounts employed. The heterocycle-containing compound may be incorporated into sustained-release preparations and devices.

The heterocycle-containing compounds described herein can be incorporated directly into the food of the subject's diet, as an additive, supplement, or the like. Any food is suitable for this purpose, although processed foods already in use as sources of nutritional supplementation or fortification, such as breads, cereals, milk, and the like, may be more convenient to use for this purpose.

### Methods of Use

The present invention is further directed to methods for treating certain conditions in a subject as well as various in vitro methods. The conditions include, for instance, intestinal fluid loss, whooping cough, anthrax, and smooth muscle contraction, and are described in greater detail herein. The methods include administering a composition including a heterocycle-containing compound to a subject who is at risk of developing or has developed one of the conditions. As used herein, the term “subject” includes humans, agriculturally important animals such as cows, pigs, poultry, sheep, and horses, as well as other animals (for instance, mice, rats, dogs, cats, and rabbits) that can be used as animal models in the study of the conditions described herein.

Treatment of the conditions described herein can be prophylactic or, alternatively, can be initiated after the development of a condition described herein. Treatment that is prophylactic, for instance, initiated before a subject manifests symptoms of a condition described herein and/or before exposure to a pathogen associated with (i.e., caused by) one of the conditions described herein, is referred to herein as treatment of a subject that is “at risk” of developing the condition. Accordingly, administration of a composition can be performed before, during, or after the occurrence of the conditions described herein. Treatment initiated after the development of a condition may result in decreasing the severity of the symptoms of one of the conditions, or completely removing the symptoms. Non-limiting examples of subjects particularly suited to receiving the composition are those undergoing antibiotic treatment, in particular the elderly and the very young, preferably antibiotic treatment that has been associated with antibiotic-associated colitis, those traveling to a location where pathogens causing intestinal fluid loss are endemic (for instance, those likely to contract Traveler's diarrhea), and those infected with HIV.

A composition that is administered to a subject who has or is at risk of developing a condition described herein includes an effective amount of a heterocycle-containing compound, preferably, a heterocycle derivative, and for certain embodiments, a diphenyl-substituted heterocycle derivative and/or a prostaglandin analog. As used herein, an “effective amount” is an amount effective to decrease or prevent (for prophylactic treatment) in a subject the symptoms associated with a condition described herein.

An aspect of the invention is directed to a method of treating intestinal fluid loss in a subject. As used herein, the term “intestinal fluid loss” refers to various types of diarrheas (i.e., an increased frequency and/or liquidity of fecal discharges when compared to normal individuals with formed stools). Intestinal fluid loss can result from, for instance, increased fluid secretion (e.g., water and/or electrolytes) from intestinal cells into the intestinal lumen, decreased absorption of water and/or electrolytes from the intestinal lumen, and/or movement of blood and mucus into the intestinal lumen. Intestinal fluid loss is usually associated with the presence of a pathogen, although foods having hyperosmolality can elicit hypersecretion of water and electrolytes. This is in contrast to idiopathic inflammatory bowel disease, which includes Crohn's disease and ulcerative colitis. The latter chronic diseases are not associated with any particular infectious agent and result from uncontrolled inflammation of the colon and other regions of the intestinal tract.

Pathogens that cause intestinal fluid loss include pathogens that are present in the intestinal lumen (for instance, Vibrio cholerae) or present in intestinal cells (for instance, Shigella), and pathogens that may not be present in the intestinal lumen or in intestinal cells (for instance, HIV). Examples of pathogens include viruses, parasites, and bacteria (see, for instance, Cotran et al., Robbins Pathologic Basis of Disease, 5^{th} ed., W.B. Sanders Co., Philadelphia, pp. 328-335 (1994)). Intestinal fluid loss caused by pathogens is referred to in the art in numerous ways, including, for instance, diarrhea, dysentery, Travelers' diarrhea, and scours (in calves).

Viruses that are associated with intestinal fluid loss include enteric viruses (for example, rotaviruses, enteric adenoviruses, and Norwalk-like viruses), and HIV. Enteric viruses typically invade and destroy mature host epithelial cells of the middle and upper villus, which causes intestinal fluid loss by the decreased absorption of sodium and water from the intestinal lumen. Infection with HIV often results in intestinal fluid loss. Typically, the fluid loss is associated with the presence of a pathogen that, due to depressed immunity, the subject is less able to clear from the intestine. Pathogens associated with intestinal fluid loss in a subject infected with HIV include Cryptosporidium, Isospora belli, Salmonella, Escherichia coli, Campylobacter jejuni, and Shigella. Parasites that are associated with intestinal fluid loss include Entamoeba histolytica, Entamoeba coli, Cryptosporidium, and Giardia lamblia.

Bacteria that are associated with intestinal fluid loss include Campylobacter jejuni, Yersinia (including Y. enterocolitica and Y. pseudotuberculosis), Shigella (including S. dysenteriae, S. flexneri, S. boydii, and S. sonnei), Salmonella (including, for instance, S. typhimurium and S. enteritidis), Clostridium difficile, enteropathogenic Escherichia coli (EPEC), enterohemmorhagic E. coli (EHEC), enteroinvasive E. coli (EIEC), and enterotoxigenic Escherichia coli (ETEC), and Vibrio cholerae.

Pathogens that are associated with intestinal fluid loss can be divided into two groups, those causing intestinal fluid loss by producing a polypeptide that causes the ADP-ribosylation of G_{Sα} (a 49 kDa polypeptide G protein present in intestinal cells), and those causing intestinal fluid loss but not producing a polypeptide having the ADP-ribosylation activity. As used herein, the term “polypeptide” refers to a polymer of amino acids and does not refer to a specific length of a polymer of amino acids. Thus, for example, the terms peptide, oligopeptide, protein, enzyme, and toxin are included within the definition of polypeptide. A “pathogen polypeptide” is a polypeptide produced by a pathogen. As used herein, the term “ADP-ribosylation” refers to the covalent addition of an adenosine diphosphate ribose (ADP ribose) to an amino acid of G_{Sα}. A polypeptide that catalyzes this addition has “ADP-ribosylation activity.”

Pathogens that produce a polypeptide having ADP-ribosylation activity include ETEC strains that secrete heat-labile enterotoxins and Vibrio cholerae. The polypeptide is typically referred to in the art as “enterotoxin.” Enterotoxin produced by V. cholera is often referred to as “Cholera toxin.” Pathogen polypeptides having ADP-ribosylation activity are secreted into the medium in which the pathogen is growing.

The ADP-ribosylation activity of a polypeptide can be measured by assay of the transfer of an ADP-ribose unit from nicotinamide adenine dinucleotide (NAD⁺) to an arginine amino acid in the presence of a buffer (see, for instance, Lai et al. Biochem. Biophys. Res. Commun., 102, 1021-1027 (1981)). Preferably, the polypeptide to be tested for ADP-ribosylation activity is present at a concentration of from about 1 micromolar to about 10 micromolar. Preferably, the buffer contains about 0.1 M 4-(2-hydroxyethyl)-1-piperazine ethanesulfonic acid (HEPES) buffer, at about pH 7.0, from zero to about 20% ethylene glycol, from zero to about 50 mM dithiohthreitol (DTE), about 300 μg of polyarginine, and about 41.4 mM NAD⁺ containing about 10 μCi of [¹⁴C]NAD⁺. Typically, the assay is incubated from about 1 minute to about 60 minutes at about 24° C. Reactions may be terminated by the addition of cold 10% trichloroacetic acid (TCA) and the polyarginine precipitate subsequently washed with cold 10% TCA on a glass microfiber filter. The radioactivity of the bound [¹⁴C]ADP-ribosylated polyarginine can measured in a scintillation counter. The level of ¹⁴C present in the precipitate at levels greater than the ¹⁴C present in a precipitate from a negative control indicates the polypeptide has ADP-ribosylation activity. The level of ¹⁴C in counts per minute (cpm) would vary with the concentration of enterotoxin. A typical assay has shown 0.2 μM of cholera toxin would bind 500 cpm, while 1.3 μM would bind 14,000 cpm. This assay can be used with isolated polypeptides or with polypeptides present in the supernatant of a culture. An “isolated” polypeptide means a polypeptide that has been either removed from its natural environment, or chemically or enzymatically synthesized. Positive controls that may be used include the supernatant of a culture of V. cholerae that expresses cholera toxin or an E. coli expressing enterotoxin.

Another type of intestinal fluid loss caused by bacteria is often referred to in the art as antibiotic-associated colitis, or pseudomembranous colitis. This typically occurs in subjects after a course of broad-spectrum antibiotic therapy, and occurs primarily in adults as an acute or chronic intestinal fluid loss. This condition may rarely appear in the absence of antibiotic therapy, for instance after surgery or in addition to a chronic debilitating illness (see, for instance, Cotran et al., Robbins Pathologic Basis of Disease, 5^{th} ed., W. B. Saunders Co., Philadelphia, p. 795 (1994)). Antibiotic-associated colitis is typically caused by Clostridium difficile, although other bacteria can also cause the disease.

In some aspects of the invention, when the intestinal fluid loss is not associated with a pathogen polypeptide having ADP-ribosylation activity (e.g., the intestinal fluid loss is associated with antibiotic treatment, the age of the subject, and/or infection by, for instance, a virus, a bacterium, a parasite, or a combination thereof), the heterocycle-containing compound present in the composition is a diphenyl heterocycle derivative, a prostaglandin analog, or a combination thereof. Examples of diphenyl heterocycles that can be used in this aspect of the invention include celecoxib, rofecoxib, SC-560, and DuP-697. Examples of prostaglandin analogs that can be used in this aspect of the invention include PGE₂-histidine and PGE₂-imidazole. Optionally, the composition can include, in addition to these heterocycle derivatives, an effective amount of metronidazole (available under the trade designation FLAGYL, from Searle and Co.) and/or an effective amount of indomethacin (available under the trade designation INDOCIN, from Merck & Co.). Of these two, metronidazole is preferred.

In another aspect of the invention, when the intestinal fluid loss is associated with a pathogen polypeptide having ADP-ribosylation activity (e.g., the intestinal fluid loss is associated with V. cholerae, ETEC, or a combination thereof), the heterocycle-containing compound present in the composition is preferably an unsubstituted heterocyclic compound (e.g., imidazole), a diphenyl heterocycle derivative, a prostaglandin analog, or a combination thereof. More preferably, the heterocycle-containing compound present in the composition can be an unsubstituted heterocyclic compound (e.g., imidazole), a diphenyl heterocycle derivative, or a combination thereof. Examples of diphenyl heterocycles that can be used in this aspect of the invention include rofecoxib, SC-560, DuP-697, and in some embodiments, celecoxib. Preferably, the compositions do not include celecoxib for this method. Examples of a prostaglandin analog that can be used in some embodiments of this aspect of the invention include PGE₂-imidazole and PGE₂-histidine. Compositions useful in this method can include an effective amount of metronidazole and/or an effective amount of indomethacin. Of these two, metronidazole is preferred.

The invention is further directed to a method of treating whooping cough in a subject. Whooping cough is a disease of the respiratory tract caused by Bordetella pertussis. After exposure to B. pertussis, cells of the respiratory tract have increased cAMP levels. The method includes administering to a subject who has or is at risk of developing whooping cough a composition that includes an effective amount of a heterocycle-containing compound. The heterocycle-containing compound present in the composition is preferably an unsubstituted heterocyclic compound (e.g., imidazole), a diphenyl heterocycle derivative, a prostaglandin analog, or a combination thereof. The heterocycle-containing compound present in the composition is more preferably a diphenyl heterocycle derivative, a prostaglandin analog, or a combination thereof. Optionally, the composition can include, in addition to, these preferred heterocycle derivatives, an effective amount of metronidazole and/or indomethacin. Of these two, metronidazole is preferred.

Another aspect of the invention is directed to a method for treating anthrax in a subject. Anthrax is an often fatal disease caused by Bacillus anthracis. One factor expressed by B. anthracis that is important in causing disease is edema factor, an adenylate cyclase which causes tissue edema by increasing cAMP levels. The method includes administering to a subject who has or is at risk of developing anthrax a composition comprising an effective amount of a heterocycle-containing compound. The heterocycle-containing compound present in the composition is preferably an unsubstituted heterocyclic compound (e.g., imidazole), a diphenyl heterocycle derivative, a prostaglandin analog, or a combination thereof. The heterocycle-containing compound present in the composition is more preferably a diphenyl heterocycle derivative, a prostaglandin analog, or a combination thereof. The heterocycle-containing compound present in the composition is more preferably a diphenyl heterocycle derivative, a prostaglandin analog, or a combination thereof. Optionally, the composition can include, in addition to, these preferred heterocycle derivatives, an effective amount of metronidazole and/or indomethacin. Of these two, metronidazole is preferred.

The present invention provides methods for inhibiting adenylate cyclase in vitro or in vivo. The adenylate cyclase may be from a prokaryotic organism or from a eukaryotic organism. Examples of prokaryotic organisms that produce an adenylate cyclase include, for instance, Pseudomonas aeruginosa (which produces the adenylate cyclase ExoY, and is thought to play a role in acute ocular pathogenesis, see, for instance, Yahr et al., Proc. Natl. Acad. Sci. USA., 95, 13899-13904 (1998)), Bordetella pertussis (which produces the adenylate cyclase CyaA, and is thought to play a role in whooping cough, see, for instance, Ladant and Ullmann, Trends Microbiol., 7, 172-176 (1999)), and Bacillis anthracis (which produces the adenylate cyclase edema factor, and is thought to play a role in anthrax, see, for instance, Leppla, Adv. Cyclic Nucl. Prot. Phosphor. Res., 17, 189-198 (1984)). As used herein, the term “in vitro” refers to a cell-free system including, for instance, an isolated adenylate cyclase, or a cell extract containing an adenylate cyclase. The method for inhibiting adenylate cyclase in vitro includes contacting an adenylate cyclase with composition that includes an amount of an heterocycle-containing compound effective to inhibit the generation of adenosine 3′,5′-monophosphate (cAMP) from adenosine triphosphate (ATP). The adenylate cyclase may be isolated from a cell, or chemically or enzymatically synthesized. Such in vitro methods can be used in various applications, such as screening for compounds having adenylate cyclase inhibiting activity.

As used herein, the term “in vivo” refers to a cell that is present in a subject. The term “in vivo” also includes a cell that has been removed from a subject, for instance a primary cell or a cell line, and a cell present in a ligated loop. Such in vivo methods may be used in, for example, screening and efficacy analyses. A ligated loop refers to a model system known to the art that can be used to assay intestinal fluid loss caused by increased adenylate cyclase activity by a pathogen polypeptide having ADP-ribosylation activity. Typically, a portion of a mouse intestine is exposed and segments are isolated by sutures. Compounds that increase adenylate cyclase activity of intestinal cells, for instance an enterotoxin, can be introduced to a segment and the amount of fluid that has accumulated in that segment after a period of time can be determined. In addition to introducing a compound such as an enterotoxin, a composition of the present invention may also be introduced and the ability of the composition to inhibit adenylate cyclase determined.

The method for inhibiting adenylate cyclase in vivo includes contacting a cell that has been removed from a subject or is in a subject with a composition that includes an amount of a heterocycle derivative effective to inhibit the generation of cAMP from ATP. The cell includes adenylate cyclase and a pathogen polypeptide having ADP-ribosylation activity. Several conditions are associated with excessive adenylate cyclase activity and include, for instance, intestinal fluid loss as in diarrheal disease, tracheal and bronchial edema as in whooping cough, and pulmonary, gastrointestinal, and disseminated edema as in anthrax. Such conditions are described herein. The methods to inhibit adenylate cyclase can be used to treat such conditions.

In another aspect, the method for inhibiting adenylate cyclase in vivo includes contacting a cell that has been removed from a subject or is in a subject with an amount of a heterocycle derivative effective to inhibit the generation of cAMP from ATP. The cell includes adenylate cyclase, but does not include a pathogen polypeptide having ADP-ribosylation activity.

Whether a heterocycle-containing compound of the present invention inhibits adenylate cyclase can be determined by measuring activity of adenylate cyclase. This can be determined by measuring tissue cAMP and the resulting amount of fluid secreted in the ligated loop model, which is described in Example 1. The activity of adenylate cyclase may also be measured by the generation of cAMP from ATP in an in vitro enzyme assay. As used herein, the term “inhibit” means prevent, decrease, or reverse the amount of fluid secreted, or the formation of cAMP. Typically, the alpha phosphate of ATP is radioactively labeled, for instance with ³²P. This assay can occur in a buffer containing about 20 mM of HEPES buffer (about pH 7.4), about 4 mM of MgCl₂, about 0.2 mg/ml BSA, about 1 mM cAMP and about 1 mM DTT. The heterocycle derivative and commercially available adenylate cyclase (from Bordetella pertussis or other sources) are added to the buffer, and allowed to incubate at about 37° C. for about 20 minutes. The cAMP is isolated, for instance by using alumina, and amount of radioactive cAMP is determined.

For methods of inhibiting adenylate cyclase, the heterocycle-containing compound present in the composition is preferably an unsubstituted heterocyclic compound (e.g., imidazole), a diphenyl heterocycle derivative, a prostaglandin analog, or a combination thereof. More preferably, the heterocycle-containing compound present in the composition can be an unsubstituted heterocyclic compound (e.g., imidazole), a diphenyl heterocycle derivative, or a combination thereof. Examples of diphenyl heterocycles that can be used in this aspect of the invention include rofecoxib, SC-560, DuP-697, and in some embodiments, celecoxib. Preferably, methods for inhibiting adenylate cyclase include celecoxib and DuP-697. Compositions useful in this method can include an effective amount of metronidazole and/or an effective amount of indomethacin. Of these two, metronidazole is preferred.

The present invention is further directed to methods of treating smooth muscle contraction, including the contraction of the uterus during, for instance, premature labor. The methods include administering a composition to a subject who has or is at risk of developing smooth muscle contractions a composition comprising an amount of a heterocycle-containing compound effective to prevent, or control by extending to substantially full-term, a premature labor. The heterocycle-containing compound present in the composition is a diphenyl heterocycle derivative, a prostaglandin analog, or a combination thereof.

The present invention is also directed to methods for modifying inflammatory responses that are mediated by PGE₂. Prostaglandins, for instance PGE₂, and leukotrienes (for instance LTB₄), are known to arise during inflammation. In high levels, PGE₂ is pro-inflammatory because it stimulates synthesis of IL-8, while in low levels, it can be cytoprotective, because of its capacity to stimulate cytokine IL-10 production. The latter cytokine (IL-10) downregulates inflammation, while the former (IL-8) signals the infiltration of polymorphonuclear neutrophils (a type of leukocyte) into the affected tissue. PGE₂ is typically produced by a cell, for instance a damaged cell, is released by the cell and interacts with a receptor on a second cell. The second cell may be a leukocyte whose fuiction is to release substances toxic for microorganisms. These substances include reactive oxygen species (including free hydroxyls, superoxide anion, and singlet oxygen), proteolytic enzymes, and acids. While toxic to microorganisms, they are also very toxic for the host's own tissues. It is expected that the prostaglandin analogs of the present invention, preferably PGE₂-imidazole or PGE₂-histidine, will bind to PGE₂ receptors and inhibit the binding of PGE₂, and possibly other prostaglandins. It is further expected that the binding of PGE₂-imidazole or PGE₂-histidine to a PGE₂ receptor will not cause a response in the cell that includes the receptor. Examples of conditions that can be treated by modifying inflammatory responses that are mediated by PGE₂ include, for instance, colibacillosis and mastitis in cattle, pancreatitis, Barrett's esophagus, gastroesophageal reflux disease syndrome (GERDS), and hepatitis.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Cholera Toxin Induced PGE₂ Activity is Reduced by Chemical Reaction with L-Histidine

Materials and Methods

Reagents. Cholera toxin (CT) and L-histidine (HCl) were purchased from Sigma Chemical Company (St. Louis, Mo). The 175-millimolar (mM) solution of L-histidine (pH 7.0) was freshly prepared for injection by adjusting to 300 milliosmoles (mosmol) with NaCl before sterilization with a 0.2 micrometer (μm) filter. Imidazole, 1-methyl-L-histidine, and 3-methyl-L-histidine were purchased from Sigma Chemical Co. (St. Louis, Mo.), and U-[¹⁵N]-imidazole was from Cambridge Isotope Laboratories (Andover, Mass.). A phosphate buffered saline (PBS) solution was made with 137 mM NaCl, 2.7 mM KCl, 1.2 mM Ca₂Cl·2H₂O, 0.49 mM Mg₂Cl·6H₂O, 8.1 mM Na₂HPO₄, and 1.47 mM KH₂PO₄ (pH 7.4).

Mouse intestinal loop assay. Adult female Swiss-Webster mice (6-8 week old) were purchased from Taconic Farms, Inc. (Germantown, N.Y.) and housed in a specific pathogen-free animal facility at UTMB in Galveston, Tex. Mice were given water without food for 18 hours before surgery to reduce the food content of the small intestine. A ventral midline incision was made under halothane anesthesia to expose the small intestine. A single 5 centimeter (cm) segment of small intestine, ligated with “00” silk suture, was constructed in each mouse. After 6 hours observation, the animals were euthanized by cervical dislocation, and the intestinal loops were removed. The amount of luminal fluid was measured and expressed as microliter per centimeter (μl/cm). Intestinal challenge was accomplished by injecting 1 microgram (μg) CT with or without 175 mM L-histidine in 100 microliters (μl) of PBS, followed by intraperitoneal injections (100 μl) of 175 mM L-histidine at the time of challenge and every 2 hours thereafter until the experiment was terminated at 6 hours. In other experiments, the dose of L-histidine was varied and administered by either the luminal or the intraperitoneal route at the time of challenge and at various times thereafter. Fluid accumulation and cell culture data (see below) were analyzed by a two-tailed Student's T test for independent samples or by Dunnett's Multiple Group Comparison test (Epistat Services, Richardson, Tex.).

Cell culture assay. The inhibitory effect of L-histidine on PGE₂ stimulation of adenylate cyclase activity in Chinese hamster ovary (CHO) cells was measured with a cAMP ELISA. CHO cells (4×10⁵) were plated in 35-millimeter (mm) dishes in Ham's F12 medium containing 10% fetal bovine serum (FBS). After overnight incubation at 37° C. with 5% CO₂, the attached cells were covered in 2 ml of fresh medium, with or without L-histidine solution (4.7 mM). All cells were stimulated with CT at indicated concentrations for 6 hours.

Ion transport studies. The inhibitory effect of L-histidine on PGE₂-stimulated sodium transport was estimated from the short-circuit current in Ussing chambers fitted with Xenopus laevis epidermis. Ussing chambers can be used to assess the effect of PGE₂-imidazole, PGE₂-histidine, L-histidine, and other compounds on Cl⁻ transport across epithelia or confluent monolayers of polarized intestinal epithelial cells (e.g., Caco-2) growing on transparent membrane inserts mounted in Ussing chamber units. The tissue or cells can be stimulated with varying concentrations (10-1000 ng/ml) of bacterial enterotoxins, including CT, E. coli STa, E. coli STb, or E. coli LTs (I and II). These protein toxins were selected because some increase cAMP levels (e.g., CT and LTs), while others increase cGMP levels (STa). In these studies, cells were grown in DMEM supplemented with 10% fetal calf serum, L-glutamine, and penicillin/streptomycin at 37° C. in 5% CO₂. Cells were seeded at a density of 0.5×10⁶ cells/ml and grown on 1 cm² PET track-etched, transparent, 0.4-mm membrane inserts (Falcon). Confluency of the monolayer was achieved when resistance reaches 200 Wcm² as determined with a volt-ohmmeter (EVOM, World Precision Instruments). Alternatively, epithelial tissues can be stretched across the chambers and used directly to assess Cl⁻ ion transport. Filters fitted with epithelia or confluent monolayers of cells were placed into a Ussing Chamber (World Precision Instruments) as described by Beltinger et al. (Amer. J. Physiol., 276, C848-C855 (1999)), and monolayers were equilibrated for 30 minutes before stimulation. Monolayers were incubated with medium containing CT (10-1000 ng/ml) or the other enterotoxins (STa, STb, and LTs) in the presence or absence of PGE₂-histidine (5 mg/ml) or CT+PGE₂-imidazole (5 mg/ml). Controls include medium alone and medium containing PGE₂-histidine, PGE₂-imidazole, or other inhibitory drugs. Using a dual-voltage clamp (World Precision Instruments), basal short-circuit current (SCC mA/cm²) and resistance (Wcm²) was determined. The stimulating enterotoxins and the PGE₂ adducts were added to either the basolateral or apical surface and changes in SCC are determined.

PGE₂ (Sigma Chemical Co.) was diluted to a concentration of 1 micromolar (μM) and incubated for 30 minutes with 10 mM L-histidine before adding to the chambers at 37° C. A reduction in short-circuit current I_{SC} was an indication that L-histidine had altered the biological activity of PGE₂. The principal solution used in the Ussing chamber studies was a NaCl Ringer solution composed of 90 mM NaCl, 2.5 mM KCl, 1.0 mM MgCl₂, 0.5 mM NaH₂PO₄, 1.8 mM CaCl₂, and 10.0 mM Hepes. A tetramethyl-ammonium chloride (TMA-Cl) Ringer solution was used, in which the NaCl was replaced by 90 mM TMA-Cl, keeping KCl, CaCl₂, and Hepes at the same concentrations as the NaCl Ringer. A 10-mM L-histidine solution was prepared with the same components and concentrations as the NaCl Ringer, except the concentration of NaCl was reduced to 85 mM. The PGE₂ solution was made by adding 20 μl of PGE₂ dissolved in H₂O to either the NaCl Ringer or to the L-histidine solution to obtain the desired concentration of 1 μM. Each of these solutions was titrated to a pH of 7.6 and had an osmolality of 205-220 mosmol/ml.

Cyclic AMP assay. Adenosine 3′,5′ monophosphate (cAMP) was extracted from the culture supernatants and quantified by a radiometric protein kinase-binding assay described previously (Peterson et al., Toxicon., 21, 761-775 (1983)) or supernatants were assayed with a radiometric cAMP binding assay (Peterson et al., Toxicon., 21, 761-775 (1983)) or an ELISA (Biomedical Technologies, Inc., Stoughton, Mass., Catalog No. BT-730) using the manufacturer's suggested procedure. The ELISA is based on the competitive binding by cAMP and an alkaline phosphate derivative of cAMP for a limited amount of antibody. The amount of enzyme-labeled cAMP bound to antibody decreases with increasing concentration of cAMP.

Reaction of PGE₂ with imidazole. Structural analysis of PGE₂-imidazole by mass spectroscopy and NMR was facilitated by adding U-[¹⁵N]-imidazole to reaction mixtures, which were incubated at 37° C. for various periods of time up to 24 hours. Some reactions were performed using 2.5 μCi of [5,6,8,11,12,14,15³H]-PGE₂ (Amersham Radiolabeled Chemicals, St. Louis, Mo.) in lieu of PGE₂. Reaction mixtures were prepared by combining 5 mM PGE₂ (Sigma Chemical Company) with 58 mM imidazole or U-[¹⁵N]-imidazole. In order to maintain the pH at 7.4, reaction mixtures contained concentrated (3.3×) PBS (457 mM NaCl, 9 mM KCl, 4 mM CaCl₂·2H₂O, 1.6 mM Mg₂Cl·6H₂O, 27 mM Na₂HPO₄, and 4.9 mM KH₂PO₄). None of the PBS components was essential for the reaction, since adduct formation occurred at 37° C. when the pH of the aqueous solution was manually adjusted to neutral pH with 0.01N NaOH and the 24-hour crude reaction mixtures were analyzed by mass spectrometry.

Reverse-phase chromatography. Covalent adducts of PGE₂ and L-histidine/imidazole were isolated by reverse-phase chromatography on a C18 (Serva, Paramus, N.J.) column (4.6×250 mm) equilibrated with 26% acetonitrile in 0.1% TFA and flowing at 1.5 ml/min. Covalent adducts of PGE₂ and L-histidine (or imidazole) were detected in the column eluate at 190 nm, and selected fractions (1.5 ml) were dried under vacuum. The molecular structures of the newly formed derivatives were characterized by mass spectrometry (MS) and nuclear magnetic resonance (NMR) spectroscopy.

NMR spectroscopy. HPLC purified samples were pooled and dissolved in 750-μl of 100% D₂O (Cambridge Isotopes, Inc.) and analyzed at 20° C. Spectral assignment of all hydrogens of the PGE₂-imidazole-adduct was afforded by 2D-autocorrelated (COSY) and 2D-totally correlated (TOCSY; 80 millisecond (ms) mixing time) spectroscopy (Bax and Davis, J. Magn. Reson., 65, 355-360 (1985); Aue et al., J. Chem. Phys., 64, 2229-2246 (1976); and Bax and Summers, J. Am. Chem. Soc., 108, 2093-2094 (1986)). The position of covalent attachment of imidazole to PGE₂ was determined by ¹⁵N-¹H inverse detected 2D-heteronuclear multiple bond correlated spectroscopy (HMBC², 90 ms mixing time). All spectra were collected on a Varian Unity-Plus 600 MHz spectrometer with external reference to HDO (4.70 ppm).

Mass spectrometry. Positive Ion Fast Atom Bombardment—Mass Spectrometry (FAB-MS) was performed on a VG Analytical ZAB-2SE high-field mass spectrometer. A cesium ion gun was used for bombardment of the sample, which was analyzed in a matrix of glycerol/thioglycerol (1:1; volume/volume (v/v)). Electrospray ionization—MS was performed with a VG Bio-Q (Quattro II upgrade) quadrupole mass spectrometer. Samples were infused in a solvent of acetonitrile:water (1:1; v/v), containing 0.1% trifluoroacetic acid at a flow rate of 10 μl/min. Daughter ion spectra were generated from the singly charged parent ions using collisionally-activated dissociation with argon as the collision gas.

Methyl esterification was performed using a reagent of either methanol:HCl (3:1; v/v) or d₃ methanol:HCl (3:1; v/v). After adding the reagent to lyophilized aliquots of the sample, the reaction was allowed to proceed at room temperature for 10 minutes and finally dried under nitrogen. Acetylation was performed on lyophilized aliquots of the sample using a reagent consisting of trifluoroacetic anhydride: acetic acid (2:1; v/v). After mixing, the reaction was allowed to proceed at room temperature for 10 minutes and finally dried under nitrogen.

Results

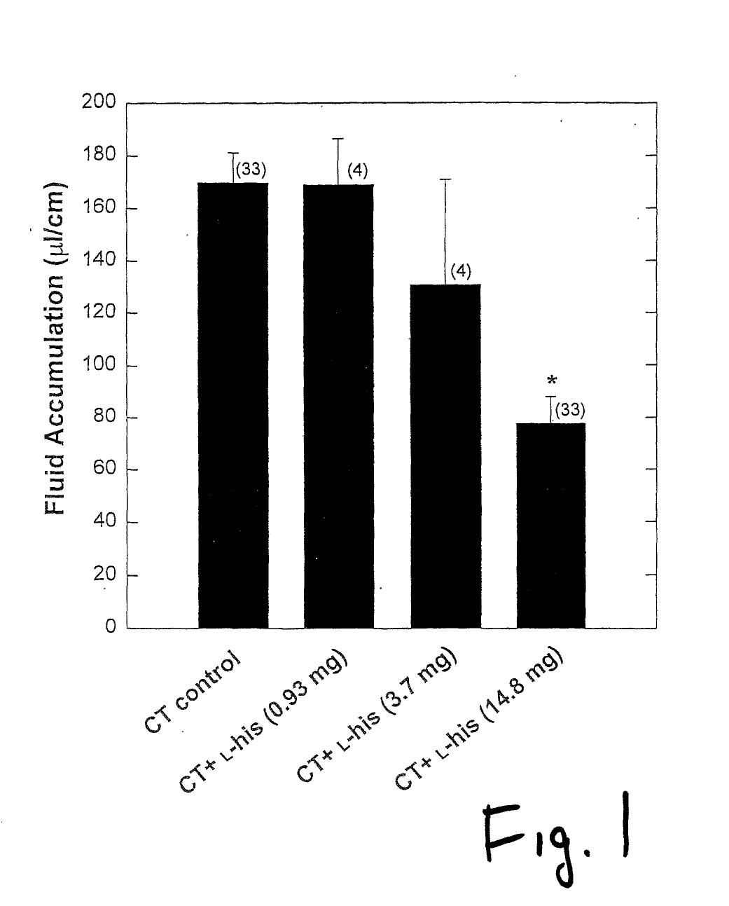

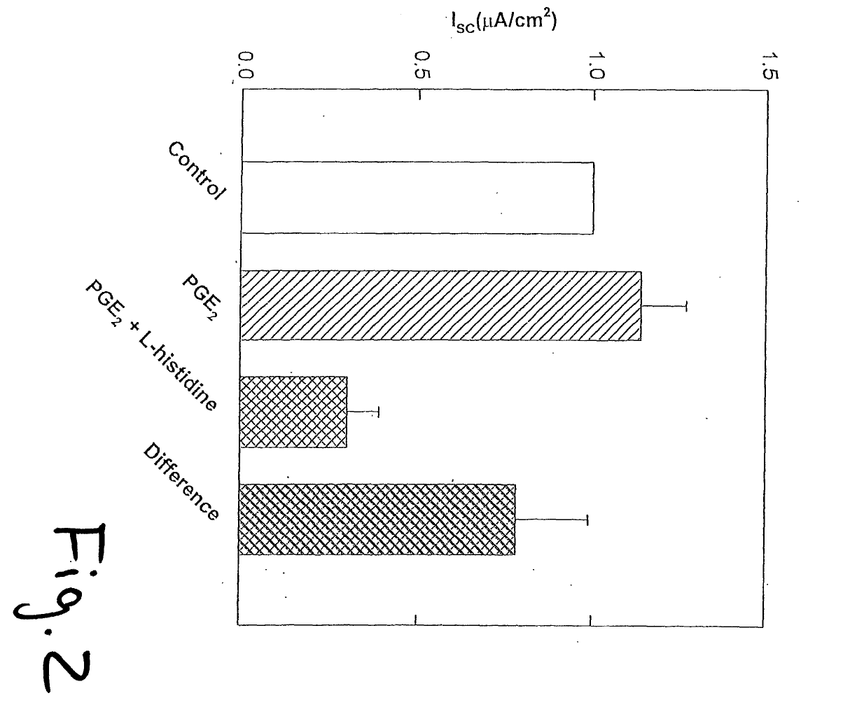

L-Histidine reduces fluid accumulation in mouse intestinal loops challenged with cholera toxin. FIG. 1 summarizes the fluid accumulation responses of control mice versus L-histidine-dosed mice challenged with CT. In this experiment, various doses of L-histidine were given to the mice during the six-hour observation period by luminal injections of 100 μl of 175, 44, or 11 mM L-histidine at the time of challenge followed by three 100-μl intraperitoneal injections of 175, 44, or 11 mM L-histidine at 0, 2, and 4 hours. The experiment was terminated after 6 hours. Since the mice received 4 injections, the total dosage of L-histidine per mouse was 14.8, 3.7, and 0.93 mg (592, 148, and 39.7 mg/kg). The results indicate that as the dose of L-histidine was increased, the amount of fluid accumulation decreased; however, statistical significance (P<0.05) was observed only at the highest dose of L-histidine tested (14.8 mg).

Effect of L-histidine on PGE₂-induced sodium transport. One possible mechanism by which L-histidine might reduce CT-induced fluid accumulation in mouse intestinal loops could be the capacity of L-histidine to chemically react with PGE₂ thereby reducing its biological activity. In vitro, L-histidine reduced both basal and PGE₂-induced sodium transport in Xenopus laevis epidermis mounted in a modified Ussing chamber (FIG. 2). PGE₂ (1 μM) increased the steady-state Na⁺-dependent current (I_{SC}) by 14% (the maximum PGE₂-induced change in I_{SC} was 18±3%, n=5, P<0.01), and 1 μM PGE₂ plus 10 mM L-histidine decreased I_{SC} to 30±9% of control (n=5, P<0.025). These data suggested an interaction between PGE₂ and L-histidine that might diminish PGE₂'s stimulatory effect on ion transport.

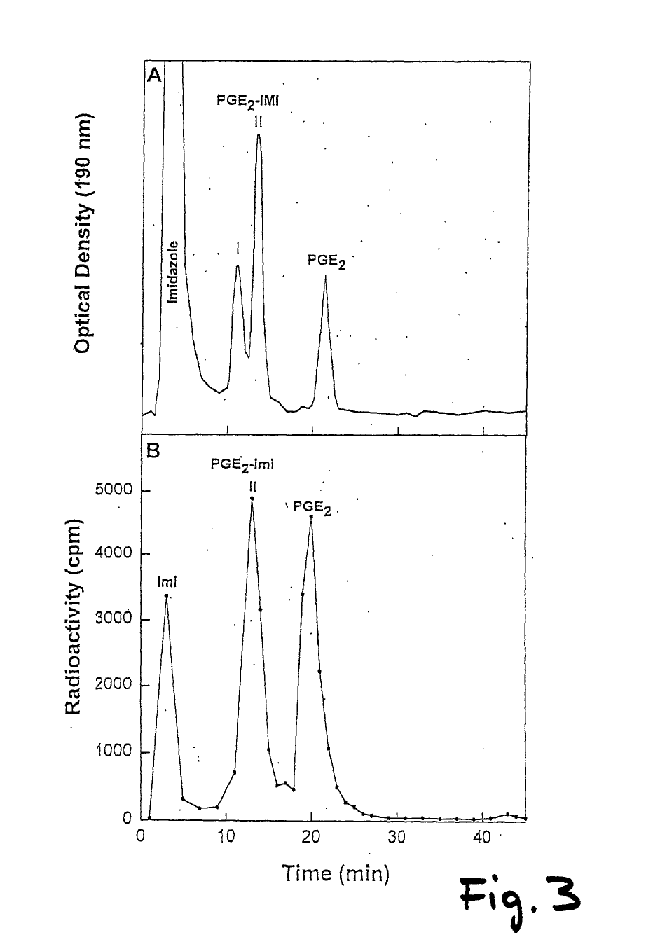

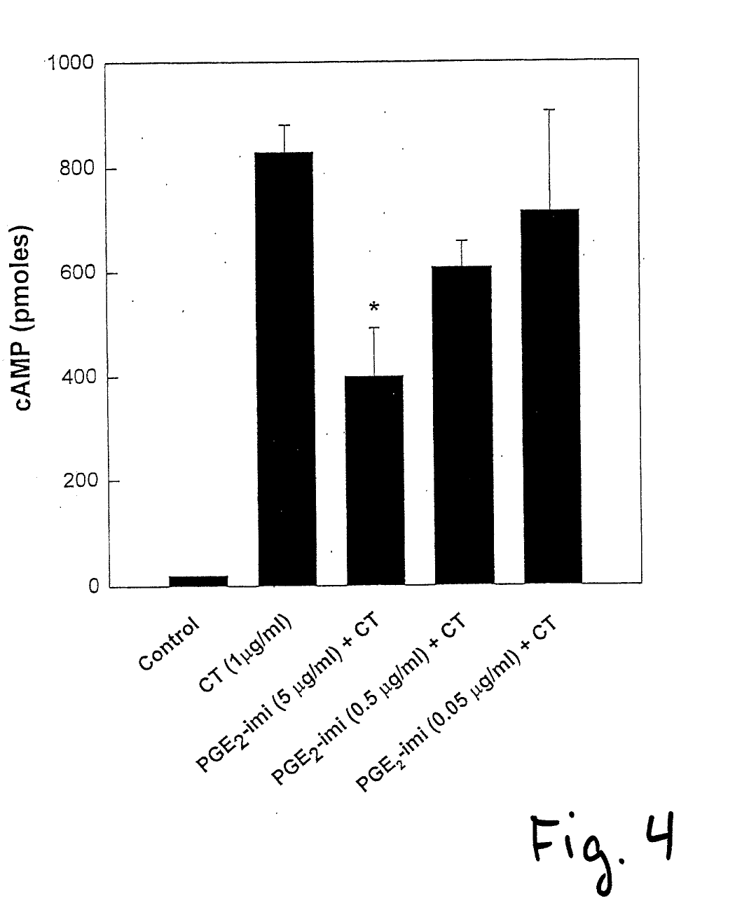

Isolation of PGE₂-imidazole/-histidine adducts. Incubation of PGE₂ and L-histidine or imidazole together under nitrogen in vitro (pH 7.4, 37° C., 24 hours) resulted in the formation of PGE₂-histidine or PGE₂-imidazole covalent adducts. These adducts were isolated by C18 reverse chromatography as illustrated for PGE₂-imidazole covalent adducts (FIG. 3A). The chromatogram was derived with a C18 reverse-phase column eluted with 26% acetonitrile in 0.1% TFA. Imidazole eluted in the void volume of the column because of its hydrophilicity. In contrast, PGE₂ eluted at 21 minutes, while PGA₂ and PGB₂ would elute about 44 and 46 min, respectively. Two new peaks appeared at approximately 10 and 12 minutes when reaction mixtures of PGE₂ and imidazole, incubated at 37° C., pH 7.0 for 24 hours, were chromatographed. L-Histidine reaction mixtures (37° C., pH 7.0, 24 hours) yielded a similar pattern except that the PGE₂-histidine peaks eluted at 8 and 9 minutes. Rechromatography of the dried fractions from peaks I and II, containing the PGE₂-imidazole adduct, eluted as a single peak comparable to peak II of FIG. 3A. Chromatography of reaction mixtures containing [³H]-PGE₂ and imidazole (FIG. 3B), containing minimal phosphate buffer, also yielded a single peak that coincided with peak II (FIG. 3A). The chromatogram was derived on the same column and with the same conditions as in Panel A. A single radioactive peak, identical to peak II (Panel A), was observed. Rechromatography of either peak I or II of PGE₂-imidazole from Panel A eluted as a single peak that coincided with the elution of [³H]-PGE₂-imidazole. A virtually identical chromatography profile was observed for the PGE₂-histidine covalent adducts, except that the two PGE₂-histidine peaks eluted at slightly earlier times (8 and 9 minutes).

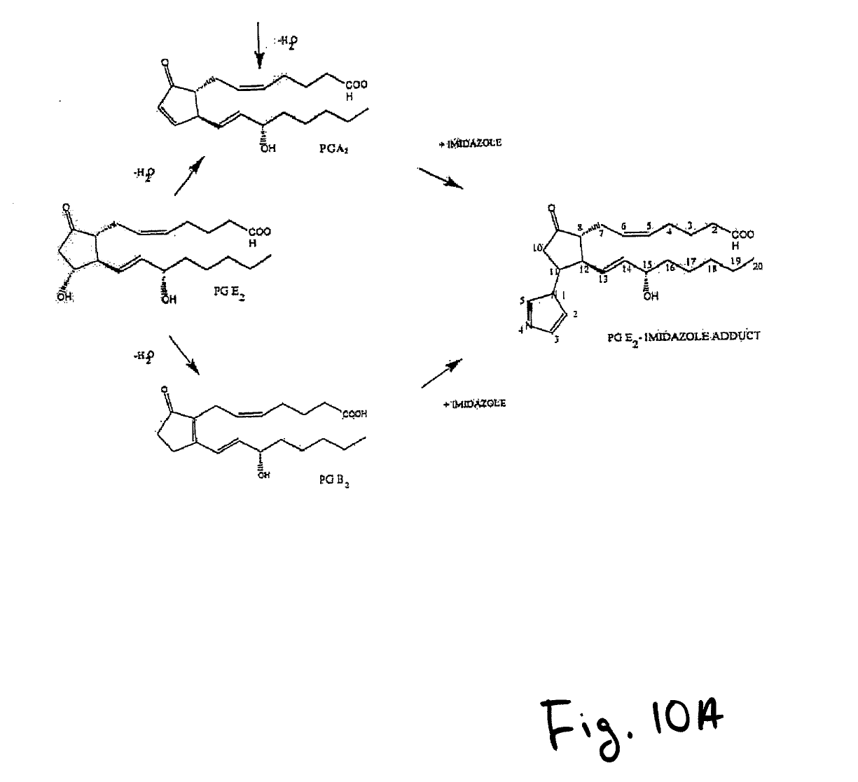

Mass spectrometry revealed the molecular weight of the two HPLC peaks containing PGE₂-histidine to be 489 Da, while the molecular weight of each PGE₂-imidazole peak was 403 Da. In a control experiment, the low pH of the HPLC buffers was not required for adduct formation, since mass spectroscopic analysis of crude mixtures of PGE₂ and imidazole (37° C., pH 7.0, 24 hours), without purification, revealed the presence of adducts. Likewise, we observed that imidazole reacted with both PGA₂ and PGB₂, which are similar in structure to PGE₂ but lack an —OH group on carbon #11 (see FIGS. 10A and 10B). The masses of the resulting PGA₂-imidazole and PGB₂-imidazole adducts by ESI-MS were the same as that of the PGE₂-imidazole covalent adduct (403 Da).

By blocking the pi or tau nitrogens in the imidazole ring of L-histidine with a methyl group, which of the two nitrogens in the imidazole ring of L-histidine reacted with C11 of PGE₂ was determined. Mixtures of PGE₂ containing either 1-methyl-L-histidine or 3-methyl-L-histidine were prepared and chromatographed. Adduct was detected when 1-methyl-L-histidine was used, because the tau nitrogen was available for covalent bonding to C11. In contrast, the tau nitrogen is blocked by the methyl group in 3-methyl-L-histidine and no adduct was formed. Thus, the tau nitrogen of L-histidine is essential for covalent bonding to C11 of PGE₂.

Effect of L-histidine/imidazole on CT-induced cAMP formation. L-Histidine reduced the capacity of both PGE₂ and CT to stimulate cAMP formation in CHO cells in vitro with a competitive cAMP-binding radiometric assay (FIG. 4). The results show the effect of purified PGE₂-imidazole adduct, isolated as in FIG. 6, on cAMP levels in CT-stimulated CHO cells. The addition of purified PGE₂-imidazole adduct to CT-stimulated CHO cell cultures resulted in significant inhibition of CT-induced cAMP formation (P<0.05). A concentration of 0.5 μg/ml reduced cAMP levels by approximately 50% in a 6-hour incubation period.

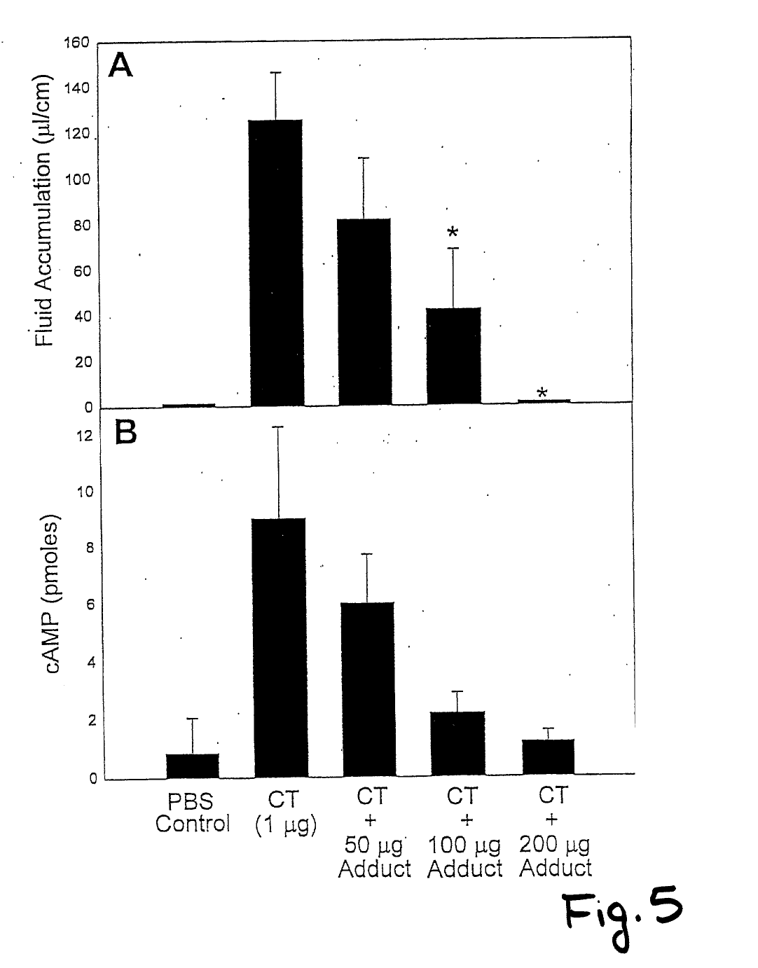

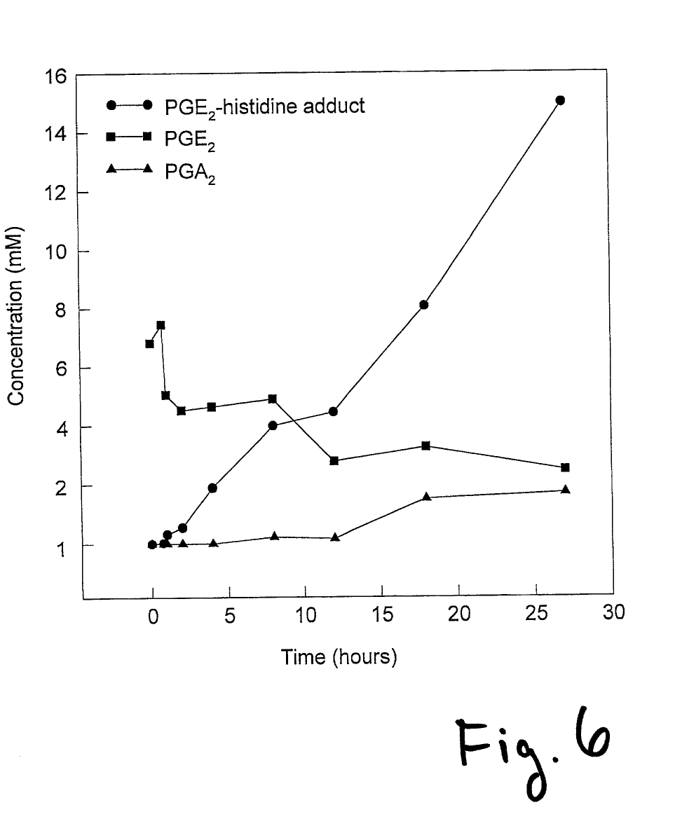

PGE₂-imidazole adduct reduces CT-induced fluid accumulation. Considering that purified PGE₂-imidazole inhibited cAMP formation in CT-stimulated CHO cells (FIG. 4), the capacity of this adduct to block CT-induced fluid accumulation in murine intestinal loops was tested. FIG. 5A shows that PGE₂-imidazole, in doses as low as 100 μg, instilled into the intestinal lumen significantly reduced CT-induced fluid accumulation. A dose of 200 μg completely blocked fluid loss following CT challenge during the 6-hour observation period. The cAMP levels (FIG. 5B) in the intestinal loop fluids were markedly reduced by PGE₂-imidazole treatment and coincided with the reduction in fluid accumulation.

Rate of PGE₂-histidine adduct formation. The rate of adduct formation was determined by measuring the relative area under the major absorbance peak at 190 nm (approximately 10-12 minutes) by C-18 reverse-phase chromatography (FIG. 3A). It was determined that the PGE₂-histidine adduct was formed in the greatest amount when the pH of the reaction mixture was 6.5 or higher. The amount of adduct formed (peak II) between PGE₂ and histidine was related to time of incubation with a T½ equal to approximately 10 hours (FIG. 6). The downward slope of the PGE₂ curve shows the consumption of PGE₂ in the reaction, while the adduct curve shows an upward slope as it increases in formation. The PGA₂ curve shows that PGA₂ is formed during the reaction due to degradation of PGE₂ or the adduct. The kinetics of PGE₂-imidazole formation was very similar to that of PGE₂-histidine.

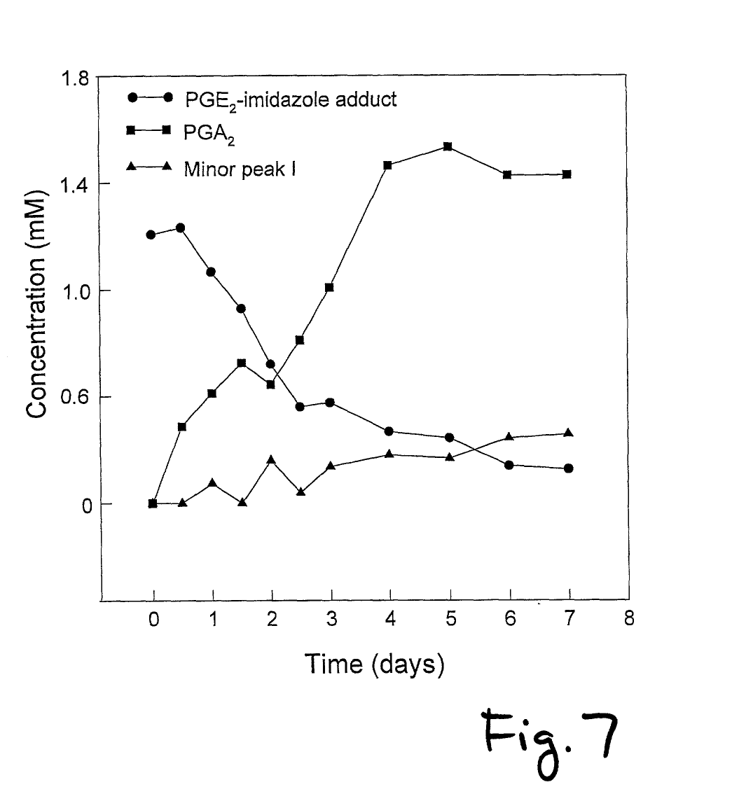

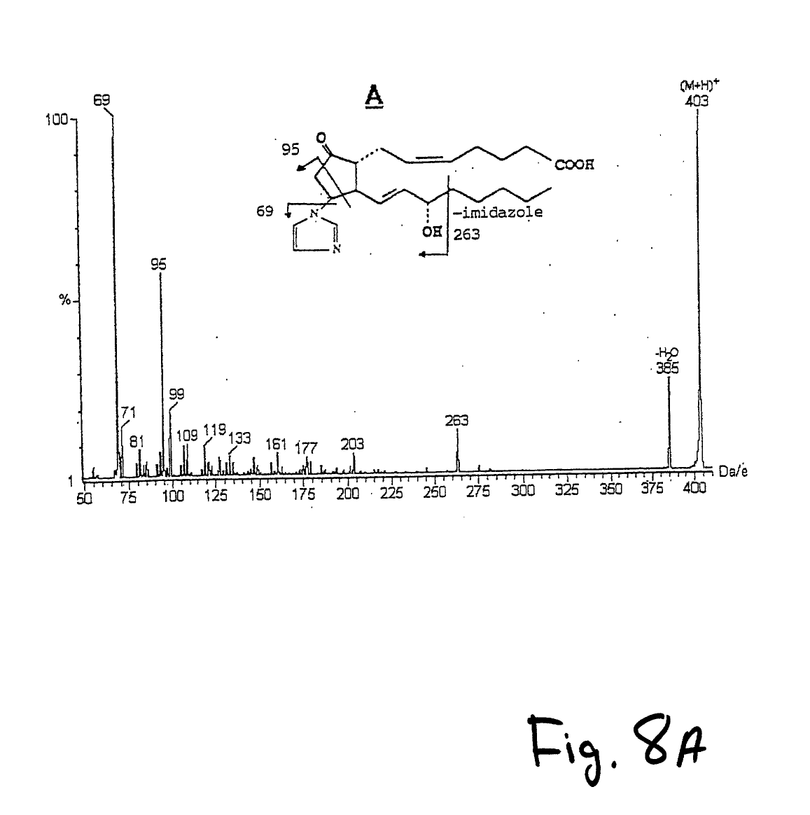

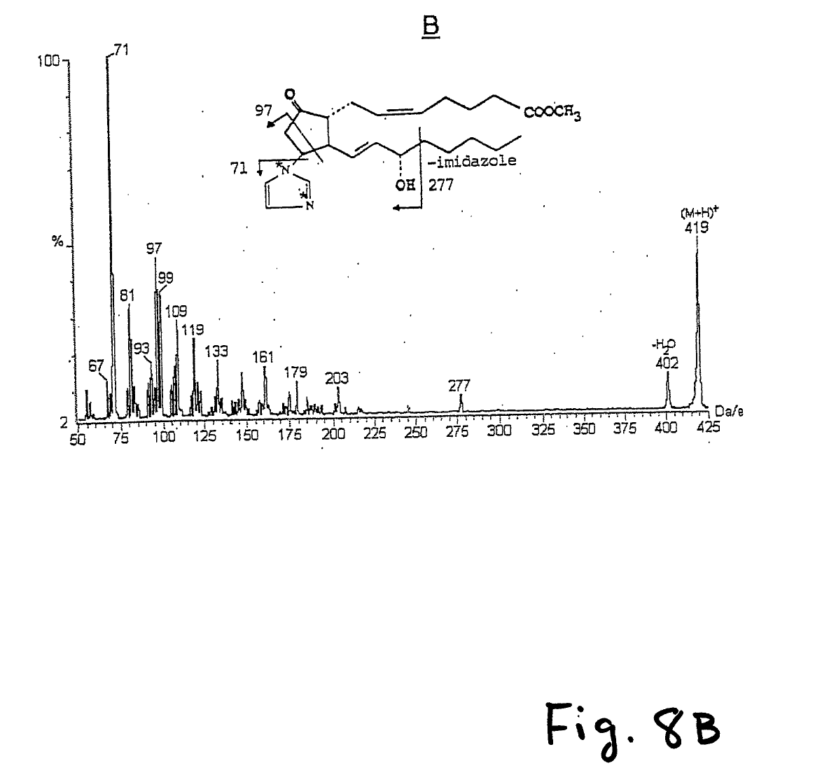

Stability of PGE₂-imidazole adduct. Purified PGE₂-imidazole adduct (peak II) was isolated by C18 reverse-phase chromatography as described in FIG. 3A and lyophilized for storage. Subsequently, 20 μg aliquots were diluted in water (200 μg/ml) and incubated at 37° C., pH 5.5 under N₂ for indicated periods of time. Samples were rechromatographed and the areas beneath each peak were integrated. The adduct appeared stable for approximately 12 hours, after which some decrease was evident by 24 hours and only 10% remained within 1 week (FIG. 7). As the adduct degraded, a peak of PGA₂ (44 min) increased in concentration, and the void volume peak containing imidazole became larger. In addition to PGA₂, a second minor peak appeared, which migrated 1-2 minutes earlier than the PGE₂-imidazole peak. The latter peak was similar to the PGE₂-imidazole adduct peak I (FIG. 3A) observed during primary chromatography of crude reaction mixtures of PGE₂ and imidazole. Neutralization of the adduct with PBS before chromatography promoted the rapid elimination of the imidazole group from the PGE₂-imidazole adduct with complete conversion within 12-24 hours. Fractions containing the adducts progressively deteriorated when stored at 4° C., but lyophilized preparations of the adducts stored under N₂ were stable at −70° C.

Mass spectrometry analysis of the PGE₂-imidazole adducts. Fast atom bombardment mass spectrometry (FAB-MS) analyses of the PGE₂-imidazole adduct isolated from either HPLC peak (FIG. 3A—peak I or peak II) showed an intense (M+H)⁺ pseudomolecular ion at m/z 403. Similar data were obtained with electrospray ionization mass spectrometry (ESI-MS). The presence of a single imidazole moiety in the adduct was confirmed by analysis of a U-[¹⁵N]-imidazole product, which gave an intense pseudomolecular ion at m/z 405. The presence of a free carboxylic acid was indicated by successful esterification of the PGE₂-imidazole adducts. This was demonstrated by the FAB-MS spectrum of the product which showed a (M+H)⁺ pseudomolecular ion at m/z 419 (methanol) and m/z 422 (d₃-methanol). Analysis of the acetylated adduct (U-[¹⁵N]-labeled) by ESI-MS showed an (M+H)⁺ at m/z 489 consistent with reaction of two acetyl groups.

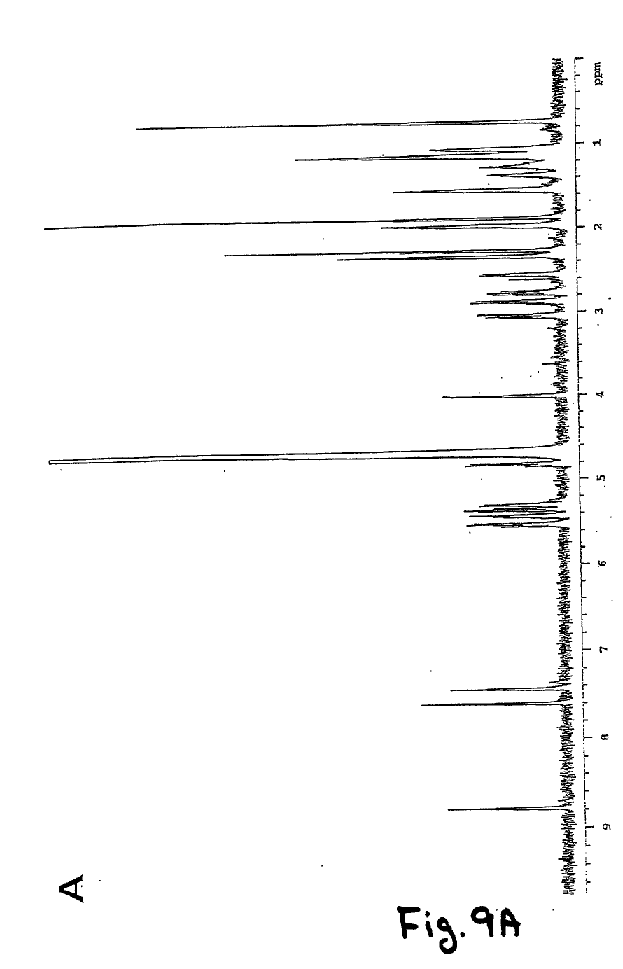

Collisionally-induced dissociation (CID) (Zirrolli et al., J. Am. Soc. Mass Spectrom., 1, 325-335 (1990)) of the PGE₂-imidazole adduct and a number of derivatives was also performed. The spectrum obtained for the PGE₂-imidazole adduct is illustrated in FIG. 8A. The major daughter ions at m/z 69 and 95 can be assigned to fragmentation of the imidazole moiety and this was confirmed by the corresponding daughter ion spectra of the U-[¹⁵N]-labeled adduct, which showed similar intense daughter ions at m/z 71 and 97 (FIG. 8B). The signal at m/z 263, which was retained in the spectrum of the U-[¹⁵N]-adduct, was consistent with a concerted fragmentation mechanism involving elimination of the imidazole and cleavage at C15. Elimination of water from the molecular ion accounted for the signal at m/z 385, whereas the low intensity ions between m/z 100-200 were consistent with cleavage along the methylene chains. FIG. 8B illustrates the ESI-MS/MS daughter ion spectrum for the esterified PGE₂-imidazole adduct and lends support to the ion assignments already given.

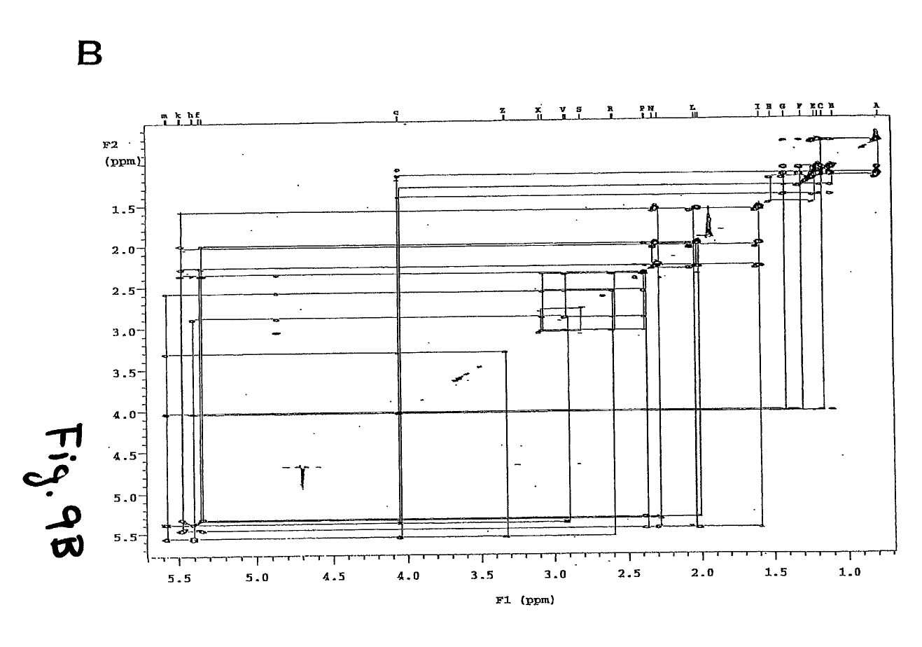

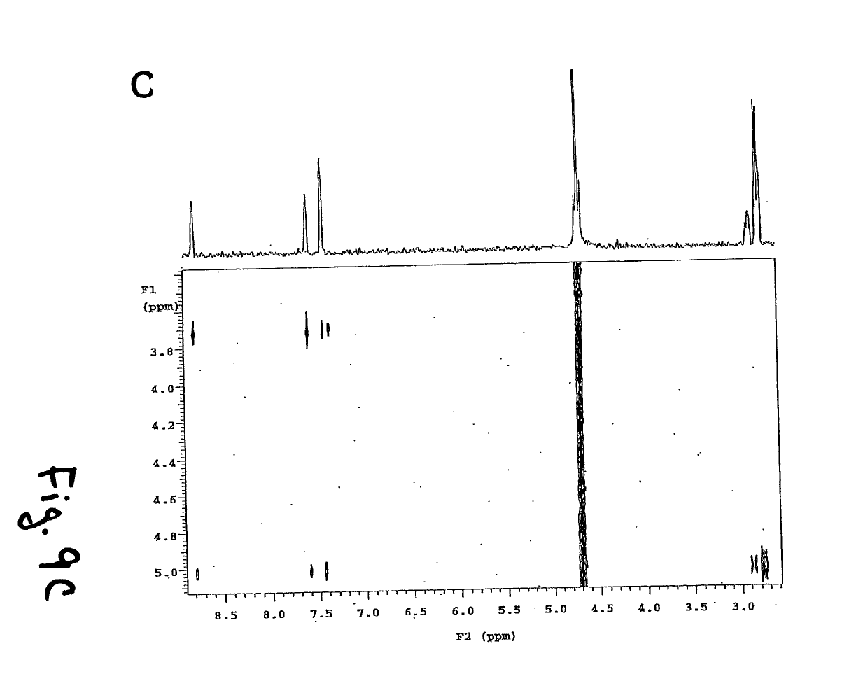

Derivation of the structure of the PGE₂-imidazole adduct by NMR. Specific information about the chemical structure of PGE₂-imidazole was derived from NMR analysis and fragmentation patterns by mass spectrometry. The 1D ¹H NMR spectrum of the PGE₂-imidazole adduct (peak II) is shown in FIG. 9A. The assignments of the ¹H signals were accomplished through analysis of the 2D COSY and TOCSY spectra (FIG. 9B). During the course of the 2D NMR spectra acquisition, some degradation of the sample was noted with several new peaks appearing. The assignments were straightforward, with cross peaks in the TOCSY spectra connecting many of the coupled protons. Thus, TOCSY correlation is seen for H-13 (5.55 ppm) to H-14, H-15 and H-12 (in order of cross peak appearance; see FIG. 10 for identification of protons). H-5 (5.45 ppm) shows correlation to H-7, H-2, H-4, and H-3. H-14 (5.37 ppm) is correlated to H-13, H-15, and H-12. H-6 (5.32 ppm) is correlated to H-5, H-7, H-2 and H-4. H-11 (4.84 ppm) shows correlation in the dimension F-2 to H-10, H-12, H-8 and H-7 (water presaturation obscures the diagonal peak and correlation in the F1 dimension). H-15 (4.03 ppm) is correlated to H-13, H-14, H-15, H-16, H-16′, H-17, and H-17′. H-10 (3.07 ppm) is correlated to H-11, H-12, H-8, and H-7. Continuing upfield, H-12, H-8, H-7, H-2, H-4, and H-3 show the expected cross peaks. Finally H-19 (1.16 ppm), H-18 (1.08 ppm), and H-20 (0.76 ppm) show correlation to each other as well as H-15 and H-16, thus completing the sequential connectivity of the protons of the prostaglandin adduct. The downfield imidazole ring protons were assigned through the COSY and TOCSY spectra, as well as the ¹⁵N/¹H HMBC spectrum of the U-¹⁵N-labeled imidazole PGE₂-imidazole adduct sample (FIG. 9C). The latter spectrum correlated the ¹⁵N/¹H coupled imidazole nitrogens with the imidazole protons H-2, H-4 and H-5 as well as two of the prostaglandin protons. Thus, N-1 of the imidazole (5.02 ppm) shows correlation to imidazole H-2 (8.81 ppm), H-4 (7.46 ppm), and H5 (7.62 ppm) as well as protaglandin protons H-12 (2.90 ppm) and H-10′ (2.79 ppm). Unfortunately, either because of small coupling or partial signal saturation due to the proximate HDO resonance, only a small, tentatively identified cross peak to the H-11 proton was observed. The correlation to both H-10 and H-12 (large three-bond coupling) confirms the site of covalent attachment of the imidazole ring to the prostaglandin framework. In addition the only significant chemical shift perturbations in the adduct relative to those of the free PGE₂ is found for H-11 (+0.74 ppm; +values represent downfield shift for the adduct), H-10, 10′ (+0.65 and +0.35 ppm), H-12 (+0.47 ppm), H-8 (+0.27 ppm) and H-14 (−0.19 ppm).

Discussion

Mouse intestinal loops challenged with CT and dosed with L-histidine accumulated significantly less fluid than those from the corresponding CT-challenged control mice (FIG. 1). Generally, the observed dose of L-histidine, providing mouse intestinal loops with maximum protection against CT-induced fluid accumulation, was relatively large (592 mg/kg), even when treatment was initiated at the same time as toxin challenge (FIG. 1).

C-18 reverse-phase chromatography of reaction mixtures of PGE₂ and imidazole or L-histidine revealed adjacent peaks at about 10-12 minutes (FIG. 3A). Peak I may be a less stable isomer of the adduct, because drying of peak I fractions and rechromatography of the material on the same column yields only peak II. The masses of the adducts (isomers) contained in the adjacent peaks were determined to be 403 Da for PGE₂-imidazole and 489 Da, for PGE₂-histidine. Further evidence was provided by the elution of [³H]-PGE₂-imidazole as a single peak (FIG. 3B) similar to peak II (FIG. 3A). The stability of the purified PGE₂-imidazole adduct (peak II) was examined by incubation for various periods of time in water at 37° C., pH 5.5 (FIG. 7). The half-life of the purified PGE₂-imidazole adduct under these conditions was approximately 2.5 days. As the PGE₂-imidazole adduct degraded, the imidazole group was eliminated resulting in the appearance of PGA₂. The void volume peak contained the released imidazole, although a small amount of peak I adduct was noted.

L-histidine was demonstrated to react chemically with PGE₂ (FIG. 3), and we considered the possibility that L-histidine inhibited the action of PGE₂ in murine intestinal loops challenged with CT. It was demonstrated that the purified PGE₂-imidazole adduct reduced cAMP levels in culture supernatants of CHO cells stimulated with CT (FIG. 4). It was surmised that L-histidine, as well as the PGE₂-imidazole adduct, interfered with the activity of PGE₂ in the CT-treated cells. It was not possible to measure the reduction of PGE₂ in vivo or in vitro by PGE₂-specific radioimmunoassays, since the PGE₂-histidine (or imidazole) adduct appeared to react equally well with antibodies to PGE₂. In part, L-histidine could have served as a PGE₂-inactivating compound, which provided additional support for the role of PGE₂ in CT-induced secretion of water and electrolytes in the small intestine. Additionally, the PGE₂-histidine covalent adduct could serve to inhibit the potential of PGE₂ to stimulate adenylate cyclase. Indeed, purified PGE₂-imidazole adduct inhibited CT-induced fluid accumulation in murine intestinal loops (FIG. 5A). In this case, the imidazole moiety may inactivate the native stimulatory effect of PGE₂ on ion transport, but it is likely the structural similarity of the PGE₂-adduct to PGE₂ that enables it to interfere with the action of CT-induced PGE₂ and fluid accumulation. Other PGE₂ analogs (e.g., PGA₂ and PGB₂) also reduce CT-induced fluid accumulation in murine intestinal loops with lower potency.

Another potent nucleophile, N-acetyl-L-cysteine (NAC), was tested to determine whether it would inhibit CT-induced fluid secretion. When injected I.P. every hour for 6 hours in a dose of 238 mM (100 μl), NAC (pH 7.0) had no protective effect for mice against CT-induced fluid secretion in small intestinal loops. Injection of a mixture of NAC and CT (without prior adjustment of the pH to 7.0) into the intestinal lumen blocked all intestinal fluid accumulation. NAC's effect on ion transport could have resulted from the low pH of the NAC solution. It was concluded that NAC could have damaged the CT protein toxin or decreased the viability of the small intestinal epithelial cells.

The NMR results established that the imidazole ring was covalently linked to PGE₂ at C-11, in effect, replacing the hydroxyl group at this carbon (Scheme I). Similar data were derived for PGE₂-histidine. Further, using methylated derivatives of L-histidine, it was established that it was the tau nitrogen, which is furthest away from the carbon chain, that reacted with C11 of PGE₂. The most reasonable explanation for this chemical transformation is the initial dehydration of PGE₂ (possibly general acid/base catalysis by the imidazole group) to yield PGA₂ or PGB₂ (FIG. 10A). Facile Michael-addition of the imidazole to this alpha, beta-unsaturated ketone will then yield the 11-deoxy-11-imidazolyl-PGE₂ (PGA₂). As shown by the pH dependence to the formation of this adduct, this occurs through the base-form of the imidazole. In additional experiments essentially as described herein, reaction mixtures were prepared with imidazole in which we substituted PGA₂ and PGB₂ for PGE₂. We observed that all three eicosanoids formed covalent adducts with imidazole and each had precisely the same mass (403 Da). These results support the sequence of events shown in FIG. 10A.

Prostaglandins are quite reactive species and readily undergo dehydration. Indeed it has been shown that albumin can catalyze similar dehydration reactions of the related PGD₂ prostaglandin. PGE₂ also undergoes dehydration. Isomerization of the double bond is quite common in prostaglandins, and it is possible that the initial 11-deoxy-Δ¹⁰-PGE₂ can also rearrange to the more fully conjugated PGB₂. It is quite likely that peak II (FIG. 3A) observed in the HPLC profile is either another stereoisomer of the 11-deoxy-11-imidazolyl-PGE₂ product, or the 12-deoxy-12-imidazolyl-PGB₂ formed by addition of imidazole to C-12 of the PGB₂ (FIG. 10B). It is thus noteworthy that PGB₂ forms adducts with imidazole having the same molecular weight as 11-deoxy-11-imidazoylyl-PGE₂ (FIG. 10B). Spectra of the PGB₂ adduct establish that the adduct is similar in structure to the one formed from PGE₂. Thus, this reinforces the point that either imidazole-catalyzed dehydration or base catalysis (or both) could explain the reaction between PGE₂ and imidazole.

It has been previously noted that albumin can covalently bind to various prostaglandins such as 15-keto-13,14-dihydro-PGE₂ and that one possible mechanism is through nucleophilic addition to an alpha, beta-unsaturated ketone dehydration product at C-11. The detailed NMR structure analysis described for 11-deoxy-11-imidazolyl-PGE₂ confirms that such a transformation is indeed quite possible. The ready addition of imidazole, as well as the imidazolyl ring of histidine, strongly suggests that histidine may be one of the residues responsible for the covalent attachment of proteins to PGE₂. This raises the possibility that prostaglandins may covalently modify proteins via the imidazole group of histidine, altering the activity of the protein or the eicosanoid.

### Example 2

### Inhibition of Intestinal Fluid Loss by Diphenyl Heterocycles

Mouse Intestinal Loop Assay

Adult female Swiss-Webster mice (25-30 g) were purchased from Taconic Farms, Inc. (Germantown, N.Y.) and housed in a specific pathogen-free animal facility at UTMB in Galveston, Tex. Mice were fasted for 18 hr before surgery to reduce the food content of the small intestine. A ventral midline incision was made under ether anesthesia to expose the small intestine. A single 5-cm segment of small intestine, ligated with “00” silk suture, was injected with 1 μg of cholera toxin (CT) in 100 μl. After 6 hours observation, the animals were euthanized by cervical dislocation and the intestinal loops were removed. The amount of luminal fluid was measured and expressed as μl/cm, while the tissue was prepared for light or electron microscopy. In some experiments, intestinal challenge was accomplished by injecting 100 μg of CT followed immediately with 160 μg/100 μl celecoxib (dissolved in 3% dimethylsulfoxide in phosphate buffered saline) at the time of challenge. Fluid volume was measured 6 hours after challenge. Specimens of fluid and tissue were collected at time of necropsy.

The inhibitory effect on CT-induced fluid accumulation was observed with dosages of celecoxib reported to be specific for COX-2.

Results

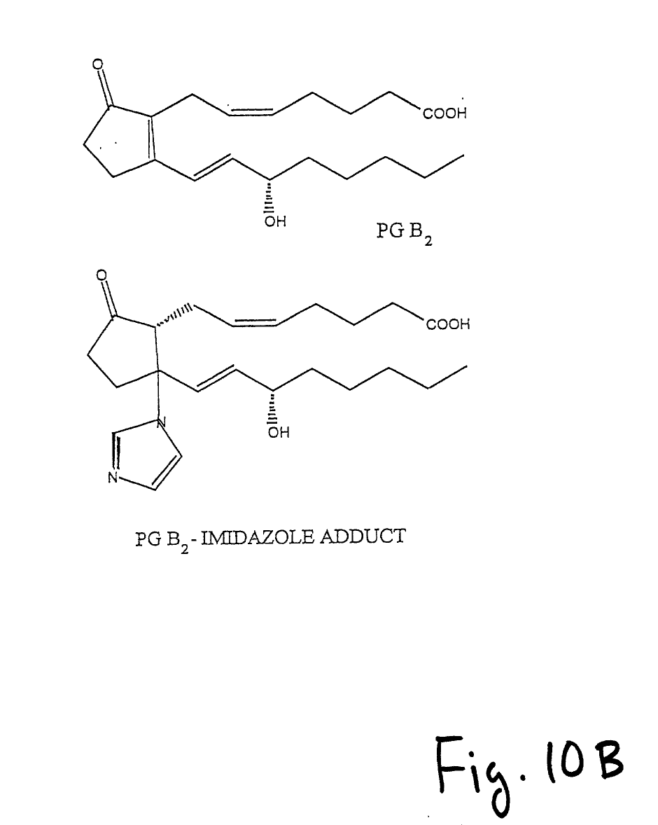

FIG. 11 shows that CT-induced fluid accumulation in murine intestinal loops is significantly reduced by celecoxib.

### Example 3

### Inhibition of Adenyl Cyclase by Heterocycle Derivatives

Assay of Adenylate Cyclase Activity.

Adenylate cyclase activity was determined by measuring the release of [³²P]-cAMP generated upon the action of the enzyme on [³²P]-ATP. The reaction is as follows:

[³²P]-ATP+Adenylate Cyclase=[³²P]-cAMP+PPi

The adenylate cyclase assay described below is similar to most other in vitro enzyme assays in that purified adenylate cyclase is mixed in a buffered solution along with the radiolabeled substrate adenosine triphosphate (³²P-ATP). Crude enzyme or eukaryotic cell membranes containing adenylate cyclase may be substituted for the purified enzyme. After incubation for 20 minutes, conversion of ³²P-ATP to product ³²P-cAMP is determined by counting the level of ³²P-cAMP formed.

Method. Substrate [³²P]-ATP (NEN, Boston Mass.) was reconstituted in the reaction buffer containing 20 mM of Hepes buffer, 4 mM of MgCl₂, 0.2 mg/ml BSA, 1 mM cAMP and 1 mM DTT, pH 7.4. A 40 μl reaction, comprised of purified adenylate cyclase (0.46 to 4.6 nmoles) (List Biological Cambell Calif.), substrate and agonist/inhibitor (1 nmole to 10 nmoles), was allowed to proceed for 20 minutes at 37° C., and the reaction was terminated with 10 μl of 0.5N HCl. The reaction mixture was transferred onto small alumina columns (Pierce, Rockford, Ill.), pre-equilibrated with 0.005N HCl and centrifuged at 500× g. The columns were washed 3× with 200 μl of 0.005 N HCl by spinning at the above speed. [³²P]-cAMP was eluted into tubes by flushing the resin 3× with 200 μl of ammonium acetate buffer. The tubes containing the eluted [³²P]-cAMP were transferred into scintillation vials. Scintillation cocktail was added, mixed and counted. [³²P]-cAMP generated was a measure of adenylate cyclase activity.

Statistical analysis. Means and standard deviations (SD) were derived from 3 values. The data were evaluated with the Student's t-test (one-tailed), and P values ≦0.05 were considered to be significantly different from controls.

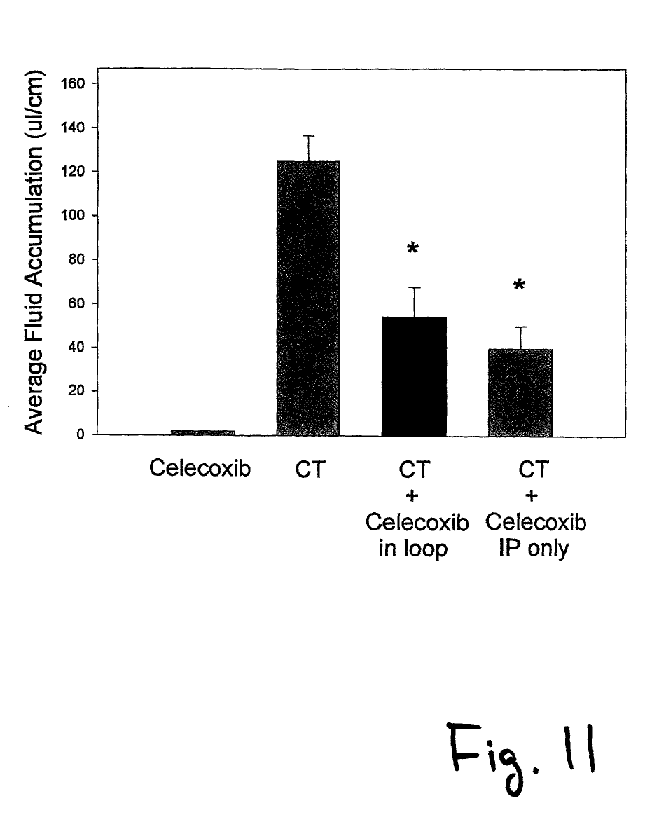

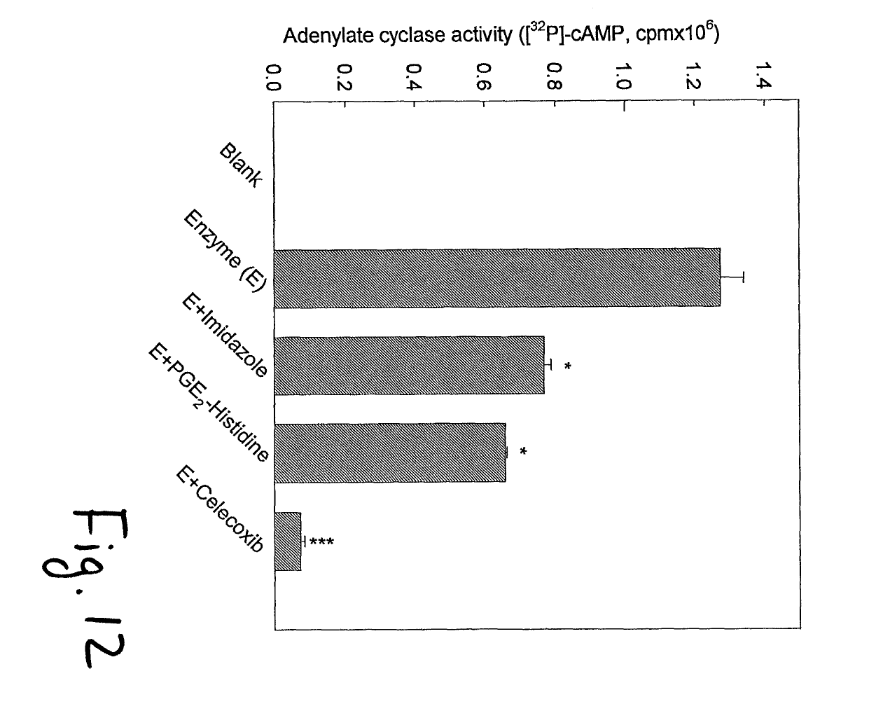

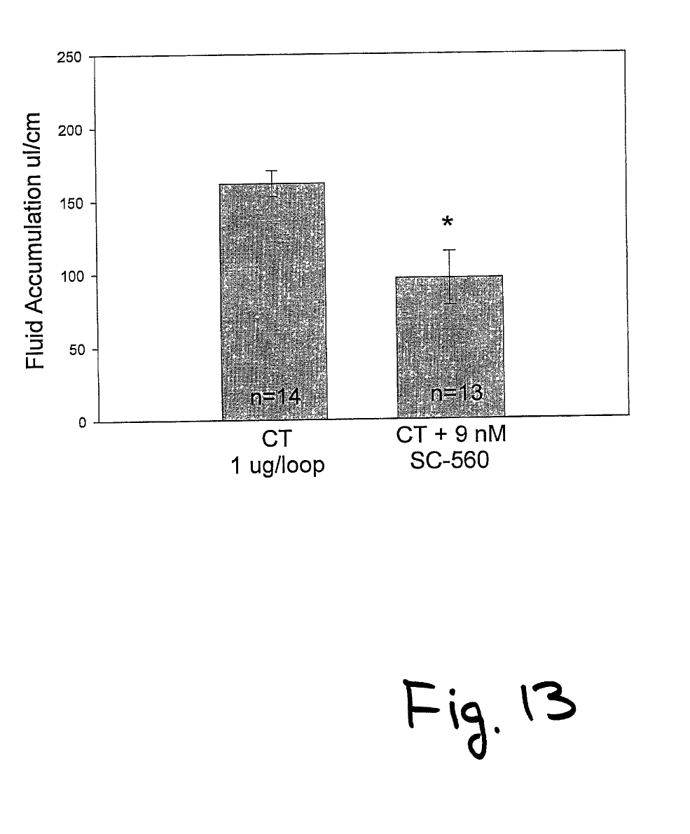

Results. The results indicate that celecoxib, PGE₂-histidine, and imidazole each inhibit adenylate cyclase enzyme activity (FIG. 12). The data in FIG. 12 also show the absence of adenylate cyclase inhibition by SC560 and rofecoxib under the conditions tested. FIG. 13 shows that SC560 inhibits cholera toxin-induced fluid secretion, although it has not been demonstrated that it does so by inhibiting adenylate cyclase under the conditions tested (FIG. 12). Rofecoxib does not inhibit cholera toxin-induced secretion under the conditions tested. Celecoxib was designed to be a highly specific inhibitor of cyclooxygenase-2 (COX-2). The mechanism by which celecoxib inhibits adenylate cyclase is not known; however, it was observed that imidazole also inhibits adenylate cyclase. Since imidazole is part of the chemical structure of celecoxib, it is suspected that this moiety participates in the functional activity of inhibiting adenylate cyclase. Imidazole is known to bind divalent cations (e.g., Mg⁺⁺, Zn⁺⁺, and Ca⁺⁺), and these metal cations are known to be required for adenylate cyclase activity. In fact, a recent report in which the X-ray crystallography-derived structure of rat adenylate cyclase was determined showed that there were two binding domains in the catalytic site of adenylate cyclase divalent cations (Zn⁺⁺ and Mg⁺⁺). We suspect that the imidazole group of celecoxib is enabling the drug to bind to the metal ions in the enzyme's active site, which would block the substrate (ATP) from entering. The end result would be inhibition of adenylate cyclase activity. From a physiological perspective in the small intestine, such an inhibitor would reduce or block cholera toxin-induced fluid loss (diarrhea).

Generation of Dose Response Curves

Methods. The adenylate cyclase enzyme assay was performed as described earlier in Example 1; however, the assay was used to assay various inhibitors (e.g., PGE₂-histidine, celecoxib, and imidazole). The amount of enzyme in each experiment was 0.46 nmole, and the concentration of each inhibitor was varied in order to determine the dose that would block 50% of the enzyme activity (IC₅₀).

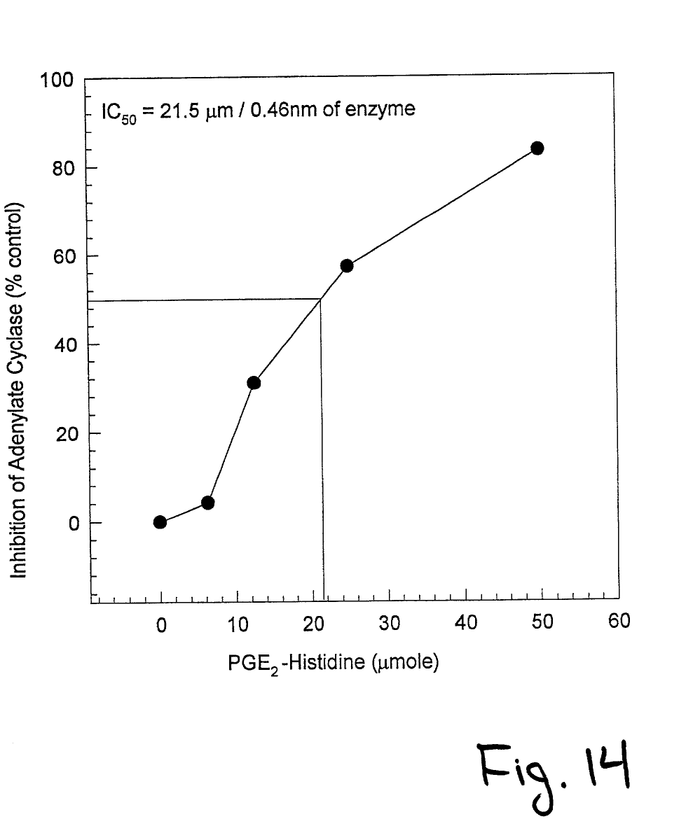

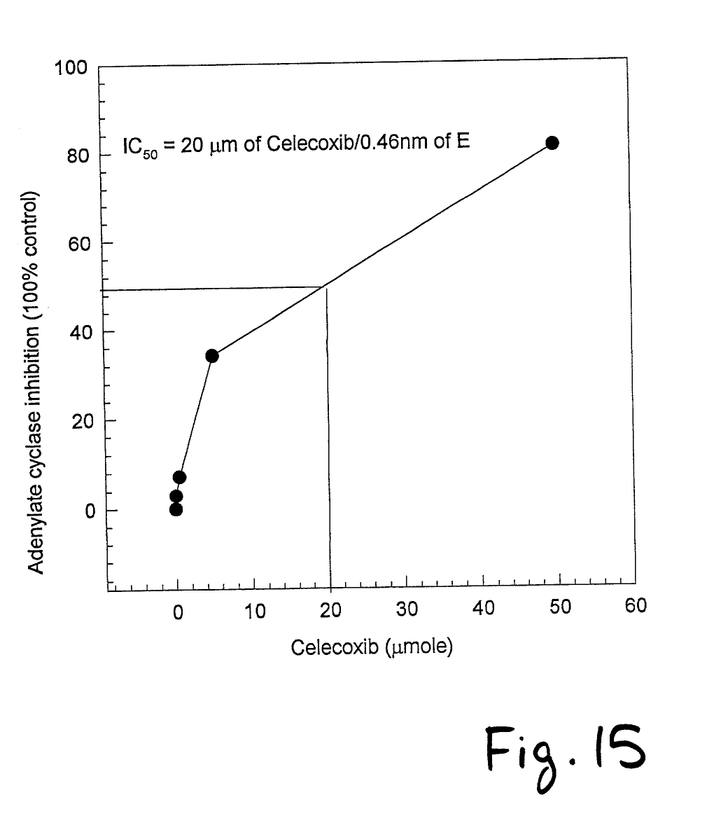

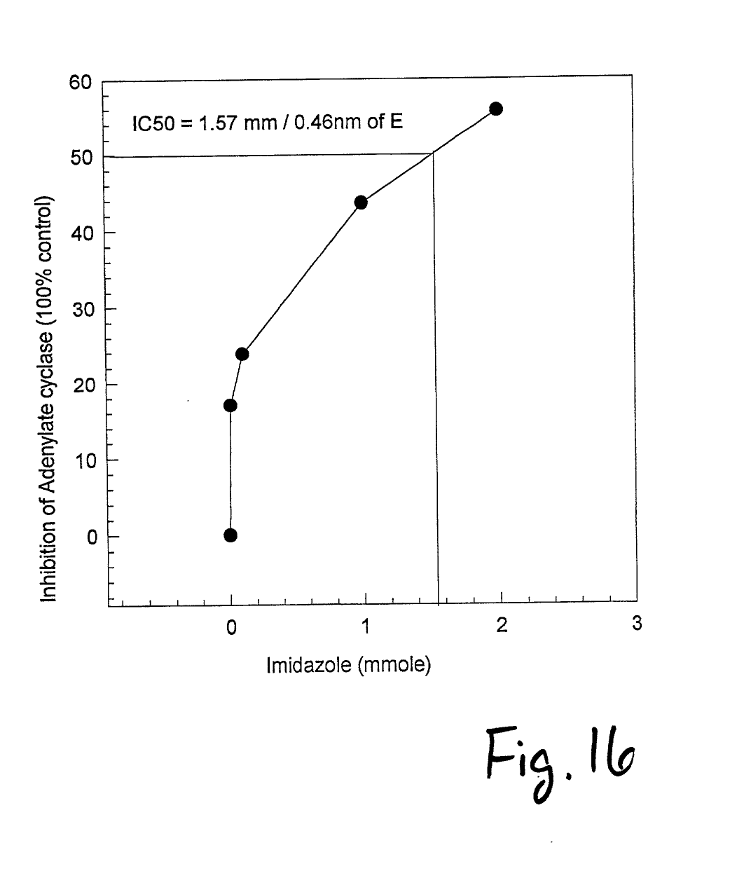

Results. The results summarized in the FIGS. 14-16 indicate that adenylate cyclase can be inhibited, which forms a strategy for reducing or blocking intestinal fluid secretion induced by several agents of diarrheal disease. FIG. 14 shows the dose response for PGE₂-histidine in inhibiting adenylate cyclase. The IC₅₀ dose of PGE₂-histidine inhibiting 50% of the enzyme activity (0.46 nmole) was 21.5 μmole. FIG. 15 shows that when a similar experiment was performed with celecoxib, and its IC₅₀ dose was 20 mmole. FIG. 16 shows that imidazole alone exhibited inhibited adenylate cyclase activity; however, it was less potent (IC₅₀=1.57 mmole). Table 1 summarizes the inhibitory potencies of the various adenylate cyclase inhibitors. Similar results were observed when edema factor from B. acthracis was used as the adenylate cyclase.

TABLE 1
Molar concentration of commonly available drugs required to
inhibit Adenylate Cyclase

| Enzyme:Drug | Ratio |
|---|---|
| Adenylate Cyclase:Celecoxib | 0.46 nm:20.0 μm |
| Adenylate Cyclase:Imidazole | 0.46 nm:1.57 mm |
| Adenylate Cyclase:Histidine:PGE₂ | 0.46 nm:21.5 μm |
| Adduct | |

### Example 4

### Inhibition of Cholera Toxin Induced Cyclic AMP Production by PGE₂

PGE₂-imidazole adduct inhibits CT-induced cAMP production in murine mucosa. One possible mechanism by which the effect on cAMP might occur is that the PGE₂ adducts might block the stimulatory effect of PGE₂ on adenylate cyclase. Experiments in which either purified PGE₂-imidazole or PGE₂-histidine was administered by intraperitoneal injection at the time of CT challenge have resulted in virtually complete inhibition of the CT fluid response (FIG. 5A). The latter experiment provides evidence against a direct effect of the adduct on the biological activity of the CT protein. The effects of the PGE₂-imidazole adduct on reducing cAMP levels in the luminal fluid from the mice is illustrated in FIG. 5B. The cAMP content of the intestinal fluid was determined by ELISA according to instructions provided by the manufacturer Biomedical Technologies, Inc., Stoughton, Mass. The latter data show that the PGE₂ covalent adduct reduces cAMP levels in the small intestine. Our studies have indicated that the PGE₂-imidazole and PGE₂-histidine adducts are not toxic for cells.

These preliminary data indicate the importance of PGE₂ in the CT-induced secretory response. Possible interpretations of these results include: 1) The similarity in structure of the PGE₂ adducts to PGE₂ may enable them to compete with PGE₂ for receptors during the intestinal response to CT, or 2) The PGE₂ adducts could also constitute competitive inhibitors of COX-1 and COX-2 enzymes. It is intriguing to consider that the PGE₂ adducts could be useful in development of future therapy against cholera and other secretory diarrheal diseases, in which the physiological effects of PGE₂ and cAMP (from adenylate cyclase) are specifically blocked.

PGE₂-imidazole adduct reduces CT-induced cAMP production in CHO cells. FIG. 4 shows that the PGE₂-imidazole adduct reduces cAMP levels in CT-stimulated Chinese hamster ovary (CHO) cells. In this experiment, HPLC-purified PGE₂-imidazole adduct was added to CHO cell cultures at the time of the challenge with CT (1 μg/ml). The cAMP content of the intestinal fluid was determined by ELISA according to instructions provided by the manufacturer Biomedical Technologies, Inc., Stoughton, Mass. Some of the resulting cAMP is formed by the toxin's capacity to ADP-ribosylate G_{Sα}, which stimulates adenylate cyclase. In addition, these data indicate that some of the cAMP arises due to the capacity of CT to stimulate the formation of PGE₂, which, in turn, stimulates adenylate cyclase. Importantly, these data show that the purified PGE₂-imidazole adduct inhibits the CT-induced PGE₂ action on adenylate cyclase.

The complete disclosure of all patents, patent applications, and publications, and electronically available material (e.g., GenBank amino acid and nucleotide sequence submissions) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method for inhibiting adenylate cyclase in vitro comprising contacting an adenylate cyclase with a composition comprising an amount of a heterocycle-containing compound effective to inhibit the generation of adenosine 3′,5′-monophosphate (cAMP) from adenosine triphosphate (ATP).

2. The method of claim 1 wherein the heterocycle-containing compound is selected from the group consisting of an unsubstituted heterocyclic compound, a diphenyl heterocycle derivative, a prostaglandin analog, or a combination thereof.

3. The method of claim 2 wherein the heterocycle-containing compound is selected from the group consisting of an unsubstituted heterocyclic compound, a diphenyl heterocycle derivative, and a combination thereof.

4. The method of claim 3 wherein the heterocycle-containing compound is an unsubstituted heterocyclic compound.

5. The method of claim 4 wherein the unsubsituted heterocyclic compound is imidazole.

6. The method of claim 3 wherein the heterocyclic-containing compound is a diphenyl heterocycle derivative.

7. The method of claim 6 wherein the diphenyl heterocycle derivative is selected from the group consisting of 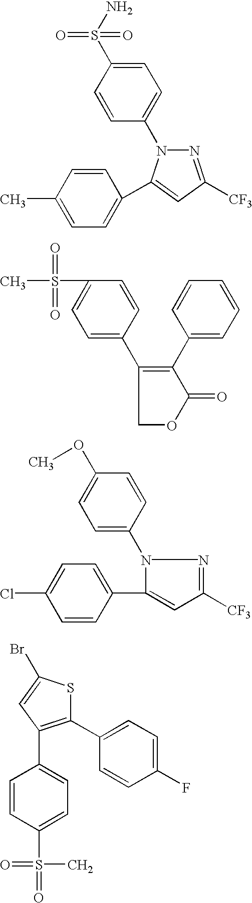
and a combination thereof.

8. The method of claim 6 wherein the diphenyl heterocycle derivative is celebrex or DuP-697.

9. A method for inhibiting adenylate cyclase in vivo comprising contacting a cell comprising an adenylate cyclase with a composition comprising an amount of a heterocycle-containing compound effective to inhibit the generation of cAMP from ATP.

10. The method of claim 9 wherein the heterocycle-containing compound is selected from the group consisting of an unsubstituted heterocyclic compound, a diphenyl heterocycle derivative, a prostaglandin analog, or a combination thereof.

11. The method of claim 10 wherein the heterocycle-containing compound is selected from the group consisting of an unsubstituted heterocyclic compound, a diphenyl heterocycle derivative, and a combination thereof.

12. The method of claim 11 wherein the heterocycle-containing compound is an unsubstituted heterocyclic compound.

13. The method of claim 12 wherein the unsubsituted heterocyclic compound is imidazole.

14. The method of claim 11 wherein the heterocyclic-containing compound is a diphenyl heterocycle derivative.

15. The method of claim 14 wherein the diphenyl heterocycle derivative is selected from the group consisting of 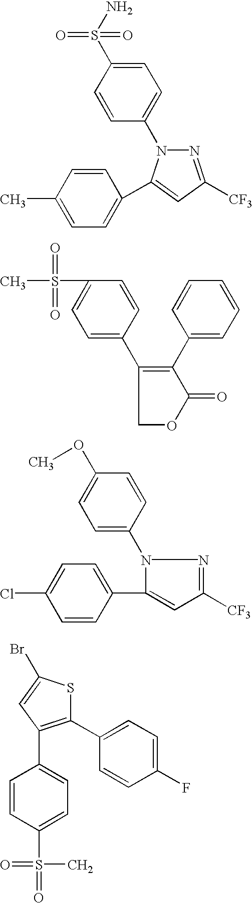
and a combination thereof.

16. The method of claim 15 wherein the diphenyl heterocycle derivative is celebrex or DuP-697.

17. The method of claim 9 wherein the cell has been removed from a subject.

18. The method of claim 9 wherein the cell is in a subject.

19. A method for inhibiting adenylate cyclase in vivo comprising contacting a cell comprising an adenylate cyclase with a composition comprising an amount of a heterocycle derivative effective to inhibit the generation of cAMP from ATP, wherein the cell does not comprise a pathogen polypeptide having ADP-ribosylation activity, and wherein the heterocycle derivative is selected from the group consisting of a diphenyl heterocycle derivative, a prostaglandin analog, and a combination thereof.

20. The method of claim 19 wherein the heterocycle derivative is selected from the group consisting of 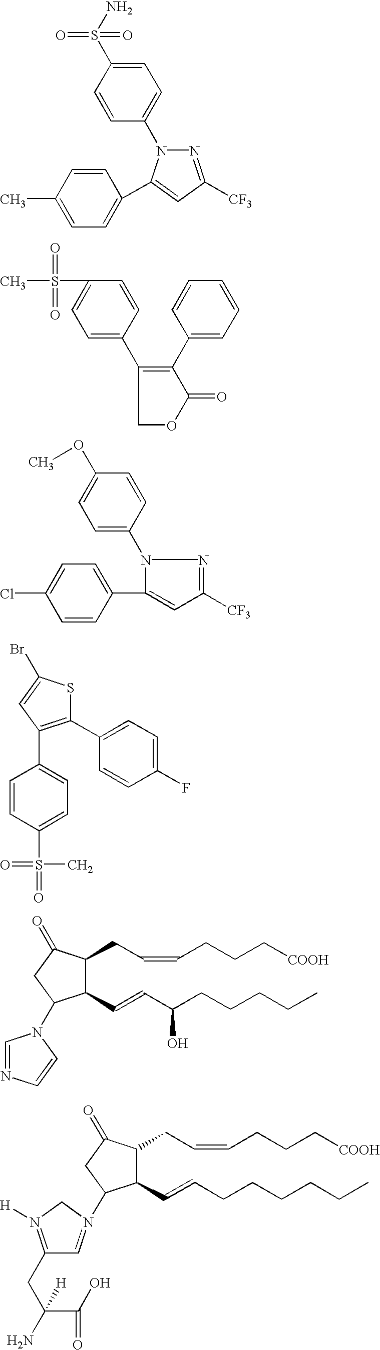
and a combination thereof.

21. The method of claim 19 wherein the cell has been removed from a subject.

22. The method of claim 19 wherein the cell is in a subject.

23. The method of claim 19 wherein the composition further includes an effective amount of metronidazole.

24. A method for treating intestinal fluid loss in a subject, the method comprising administering to a subject who has or is at risk of developing intestinal fluid loss a composition comprising an effective amount of a heterocycle derivative selected from the group consisting of a diphenyl heterocycle derivative, a prostaglandin analog, and a combination thereof, wherein the fluid loss is not associated with a pathogen polypeptide having ADP-ribosylation activity.

25. The method of claim 24 wherein the heterocycle derivative is selected from the group consisting of 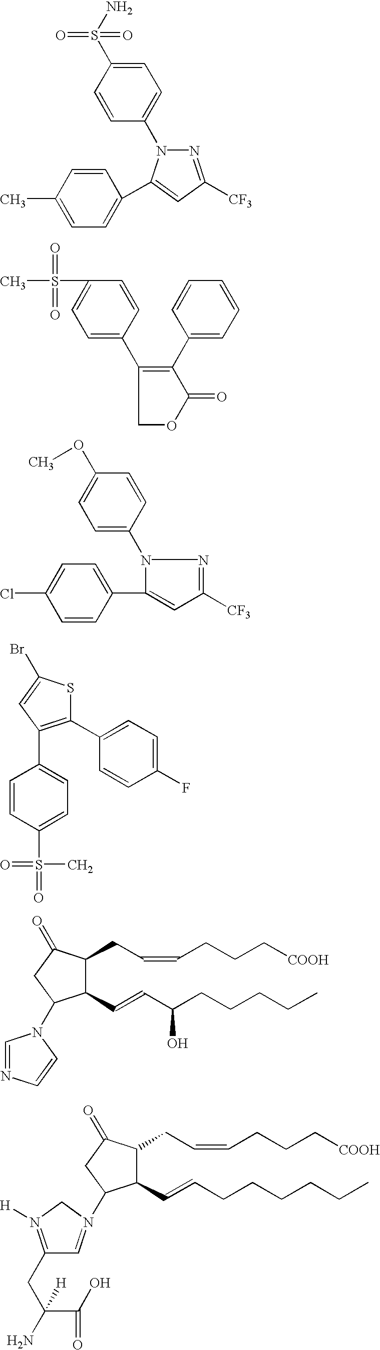
and a combination thereof.

26. The method of claim 24 wherein the composition further includes an effective amount of metronidazole, indomethacin, or a combination thereof.

27. A method for inhibiting adenylate cyclase in vivo comprising contacting a cell comprising an adenylate cyclase with a composition comprising an amount of a heterocycle-containing compound effective to inhibit the generation of cAMP from ATP, wherein the cell comprises a pathogen polypeptide having ADP-ribosylation activity.

28. The method of claim 27 wherein the heterocycle-containing compound is selected from the group consisting of an unsubstituted heterocyclic compound, a diphenyl heterocycle derivative, a prostaglandin analog, or a combination thereof.

29. The method of claim 28 wherein the heterocycle-containing compound is selected from the group consisting of an unsubstituted heterocyclic compound, a diphenyl heterocycle derivative, and a combination thereof.

30. The method of claim 29 wherein the heterocycle-containing compound is an unsubstituted heterocyclic compound.

31. The method of claim 30 wherein the unsubstituted heterocyclic compound is imidazole.

32. The method of claim 28 wherein the heterocyclic-containing compound is a diphenyl heterocycle derivative.

33. The method of claim 32 wherein the diphenyl heterocycle derivative is selected from the group consisting of 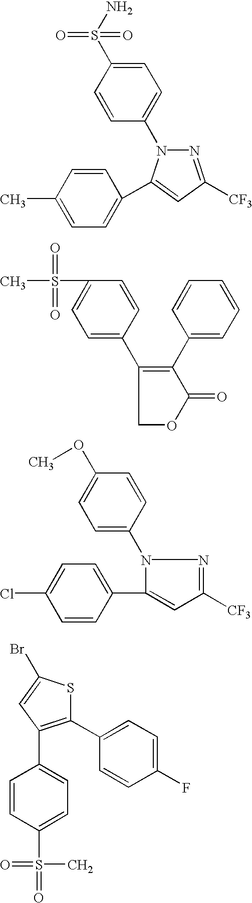
and a combination thereof.

34. The method of claim 27 wherein the heterocycle-containing compound is metronidazole.

35. A method for treating intestinal fluid loss in a subject, the method comprising administering to a subject who has or is at risk of developing intestinal fluid loss a composition comprising an effective amount of a heterocycle-containing compound, wherein the intestinal fluid loss is associated with a pathogen polypeptide having ADP-ribosylation activity.

36. The method of claim 35 wherein the heterocycle-containing compound is selected from the group consisting of an unsubstituted heterocyclic compound, a diphenyl heterocycle derivative, a prostaglandin analog, or a combination thereof.

37. The method of claim 36 wherein the heterocycle-containing compound is selected from the group consisting of an unsubstituted heterocyclic compound, a diphenyl heterocycle derivative, and a combination thereof.

38. The method of claim 37 wherein the heterocycle-containing compound is an unsubstituted heterocyclic compound.

39. The method of claim 38 wherein the unsubsituted heterocyclic compound is imidazole.

40. The method of claim 35 wherein the heterocyclic-containing compound is a diphenyl heterocycle derivative.

41. The method of claim 40 wherein the diphenyl heterocycle derivative is selected from the group consisting of 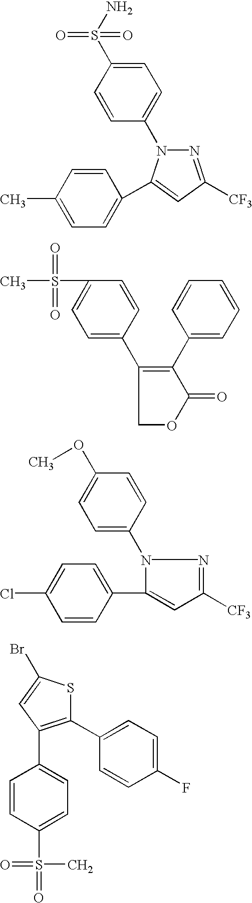
and a combination thereof.

42. The method of claim 35 wherein the heterocycle-containing compound is metronidazole, indomethacin, or a combination thereof.

43. The method of claim 35 wherein the heterocycle derivative is not celecoxib.

44. A method for inhibiting smooth muscle contraction in a subject, the method comprising administering to a subject who has or is at risk of developing a condition associated with smooth muscle contraction a composition comprising an effective amount of a heterocycle derivative selected from the group consisting of a diphenyl heterocycle derivative, a prostaglandin analog, and a combination thereof.

45. The method of claim 44 wherein the heterocycle derivative is selected from the group consisting of 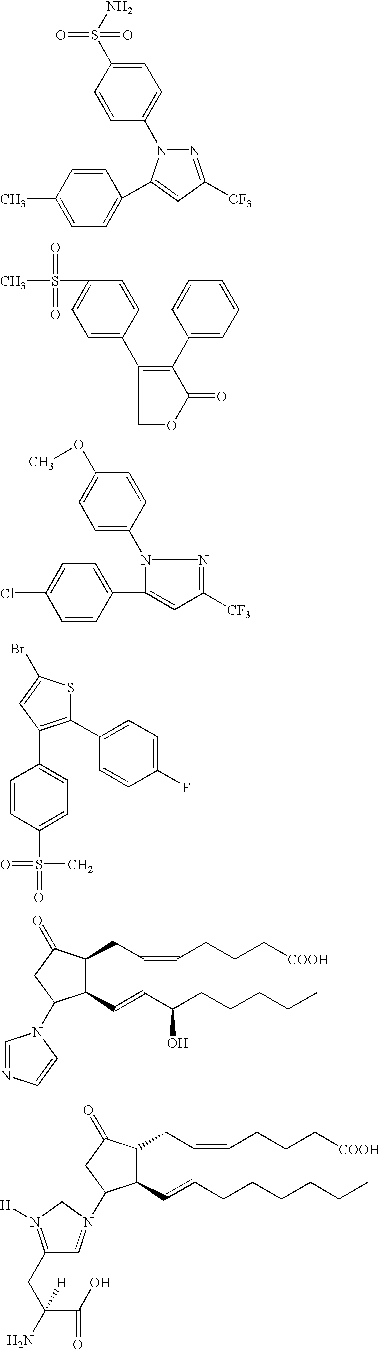
and a combination thereof.

46. The method of claim 44 wherein the composition further includes an effective amount of metronidazole, indomethacin, or a combination thereof.

47. A method for treating whooping cough in a subject, the method comprising administering to a subject who has or is at risk of developing whooping cough a composition comprising an effective amount of an heterocycle-containing compound.

48. The method of claim 47 wherein the heterocycle-containing compound is selected from the group consisting of an unsubstituted heterocyclic compound, a diphenyl heterocycle derivative, a prostaglandin analog, or a combination thereof.

49. The method of claim 48 wherein the heterocycle-containing compound is selected from the group consisting of an unsubstituted heterocyclic compound, a diphenyl heterocycle derivative, and a combination thereof.

50. The method of claim 49 wherein the heterocycle-containing compound is an unsubstituted heterocyclic compound.

51. The method of claim 50 wherein the unsubsituted heterocyclic compound is imidazole.

52. The method of claim 48 wherein the heterocyclic-containing compound is a diphenyl heterocycle derivative.

53. The method of claim 52 wherein the diphenyl heterocycle derivative is selected from the group consisting of 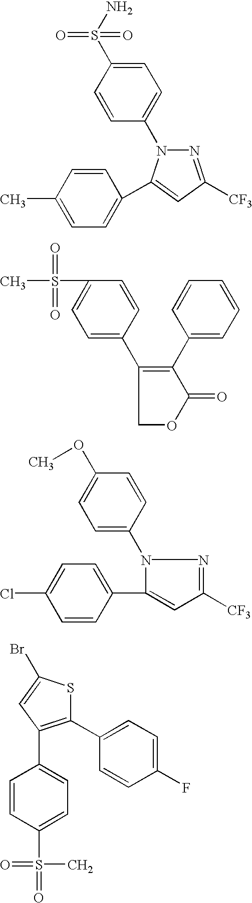
and a combination thereof.

54. The method of claim 47 wherein the heterocycle-containing compound is metronidazole, indomethacin, or a combination thereof.

55. A method for treating anthrax in a subject, the method comprising administering to a subject who has or is at risk of developing anthrax a composition comprising an effective amount of a heterocycle-containing compound.

56. The method of claim 55 wherein the heterocycle-containing compound is selected from the group consisting of an unsubstituted heterocyclic compound, a diphenyl heterocycle derivative, a prostaglandin analog, or a combination thereof.

57. The method of claim 56 wherein the heterocycle-containing compound is selected from the group consisting of an unsubstituted heterocyclic compound, a diphenyl heterocycle derivative, and a combination thereof.

58. The method of claim 57 wherein the heterocycle-containing compound is an unsubstituted heterocyclic compound.

59. The method of claim 58 wherein the unsubsituted heterocyclic compound is imidazole.

60. The method of claim 55 wherein the heterocyclic-containing compound is a diphenyl heterocycle derivative.

61. The method of claim 60 wherein the diphenyl heterocycle derivative is selected from the group consisting of 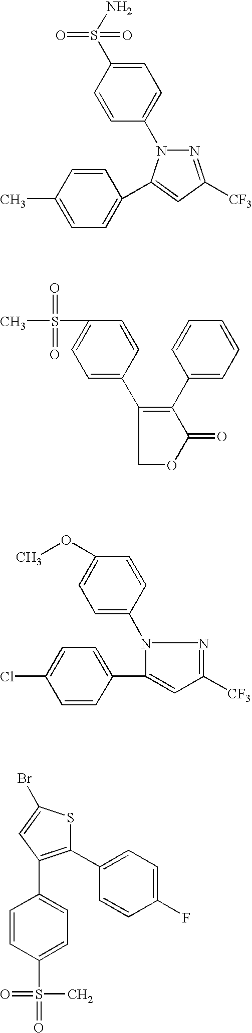
and a combination thereof.

62. The method of claim 55 wherein the heterocycle-containing compound is metronidazole, indomethacin, or a combination thereof.
